(19) United States
(12) Patent Application Publication
(10) Pub. No.: US 2024/0221950 A1
(43) Pub. Date: Jul. 4, 2024

(54) MULTI-MODAL INPUT PROCESSING

(71) Applicant: NEUMORA THERAPEUTICS, INC., Brisbane, CA (US)

(72) Inventors: Matthew KOLLADA, Brisbane, CA (US); Tathagata BANERJEE, Brisbane, CA (US)

(21) Appl. No.: 18/557,873

(22) PCT Filed: Apr. 28, 2022
(86) PCT No.: PCT/US2022/026714

§ 371 Date: Oct. 27, 2023

Related U.S. Application Data
(60) Provisional application No. 63/180,810, filed on Apr. 28, 2021.

(51) Int. Cl. G16H 50/20; A61B 5/00; G16H 10/20

(52) U.S. Cl. CPC G16H 50/20; A61B 5/0077; A61B 5/4076; A61B 5/4842; G16H 10/20

(57) ABSTRACT

The disclosed technology is directed to improvements in multi-modal and multi-sensor diagnostic devices, that utilize machine learning algorithms to diagnose patients based on data from different sensor types and formats. Current machine learning algorithms that classify a patient's diagnosis focus on one modality of data output from one type of sensor or device. This is because, among other reasons, it is difficult determine which modalities or features from different modalities will be most important to a diagnosis, and also very difficult to identify an algorithm that can effectively to combine them to diagnose health disorders.

### CROSS REFERENCE TO RELATED APPLICATIONS

This application claims priority to U.S. Provisional Application No. 63/180,810 filed Apr. 28, 2021 titled MULTI-MODAL INPUT PROCESSING, the contents of all of which are incorporated herein by reference.

### FIELD

The present invention is directed to processing data from multiple modalities for mental health evaluation.

### BACKGROUND

The following description includes information that may be useful in understanding the present invention. It is not an admission that any of the information provided herein is prior art or relevant to the presently claimed invention, or that any publication specifically or implicitly referenced is prior art.

Current approaches to mental health evaluation rely primarily on assessment by a healthcare provider. As such, accuracy of diagnosis may vary depending on experience, expertise, and/or physical and mental fatigue, among other factors. Further, other approaches largely focus on single modality processing to assist in mental health evaluation, including data preprocessing, machine learning, and diagnostic outputs based on unimodal inputs.

### SUMMARY

The disclosed technology is directed to improvements in multi-modal and multi-sensor diagnostic devices, that utilize machine learning algorithms to diagnose patients based on data from different sensor types and formats. Current machine learning algorithms that classify a patient's diagnosis focus on one modality of data output from one type of sensor or device. This is because, among other reasons, it is difficult determine which modalities or features from different modalities will be most important to a diagnosis, and also very difficult to identify an algorithm that can effectively to combine them to diagnose health disorders.

This difficulty is particularly acute in the mental health space, as mental health disorders are expressed as complex phenotypes of a constellation of symptoms that may be expressed through a patient's speech, facial expressions, posture, brain activity (e.g. EEG, MRI), cardiac activity, genotype, phenotype, proteomic expression, inflammatory marker levels, and others.

While a variety of mental health biomarkers have been proposed as single modality diagnostics, few have been able to reliably diagnose mental health illnesses. For instance, almost all current biomarkers are used alone or only in combination with the same type of biomarkers (or same type/format of data sources from the same types of sensors or devices).

The main reason most current research or products focus on a single modality is it is very complex and difficult to even determine how a single modality is relevant to an actual diagnosis of a mental health disorder. For instance, much progress has made in determining sentiment or affect, but those are much less challenging and straight forward to determine than a mental health diagnosis.

Accordingly, as multiple different biomarkers interact to relate to a mental health diagnosis—especially categorically different types of biomarkers—is extraordinarily complex. This is because mental health disorders are broad categories of illness that may encompass multiple underlying biotypes, and can exhibit different levels and types of symptoms across patients. Thus, very few have attempted to combine modalities to diagnose mental health illnesses, and none have done it effectively.

For example, the currently proposed diagnostic tools that have been described as multi-modal are primarily using the same category of data—but different types. For instance, there is some research around using “multi-modal” diagnostics for Alzheimer's disease that using different types of image data (e.g. CT and PET). But these diagnostics are not combining different categories of data, and rather only two different types of images of the brain. They are thus much easier to cross-correlate and input into a machine learning algorithm because they are both images of the same anatomical structure.

For instance, in the article “Multimodal and Multiscale Deep Neural Networks for the Early Diagnosis of Alzheimer's Disease using structural MR and FDG-PET images” published in Scientific reports, 8(1), 5697, by Lu et al., the authors combined different images to attempt to diagnose Alzheimer's Disease (“AD”). The authors found that by combining the imaging modalities they were able to increase the accuracy, but even combining these related imaging modalities was complex as described in the paper—and it only focused on identifying brain defects that are indications of AD.

Accordingly, identifying a combination of biomarkers and an algorithm that could process them in the right way to diagnose mental illness is incredibly difficult—one cannot simply plug any combination of modalities into any machine learning algorithm. For instance, an article by Strawbridge, titled “Multimodal Markers and Biomarkers of Treatment,” in Psychiatric Times, July 2018, pp. 19-20, confirms that selecting and combining biomarkers to effectively diagnosis mental health issues like depression is incredibly difficult. For instance, the author notes:
[a]lthough the findings represent potential diagnostic biomarkers, inconsistencies between studies render single biomarkers ineffectual as replacements for current diagnostic tools. Indeed, the potential for a diagnostic biomarker (or biomarkers) for depression are viewed with much skepticism, not least because it is difficult to see how they could ever outperform current diagnostic criteria.

Despite the challenges noted in the art, the inventors have developed an architecture for mental health evaluation that is capable of effectively incorporating interactions between biomarkers of mental health from multiple modalities. In one implementation, a method for evaluating mental health comprises: acquiring two or more types of modality data from two or more modalities; generating, using each of the two or more modality data, two or more sets of mental health features; combining the two or more sets of mental health features to output a combined data representation comprising outer products of the two or more sets of mental health features; and generating a mental health evaluation output according to a trained machine learning model and using the combined data representation as input.

In another implementation, a device, comprises: a first modality processing logic to process a first data modality from a first type of sensor to output a first data representation comprising a first set of mental health features; a second modality processing logic to process a second data modality from a second type of sensor to output a second data representation comprising a second set of mental health features; modality combination logic to process the first and second data representations to output a combined data representation comprising products of the first and second set of mental health features; and diagnosis determination logic to determine a mental health diagnosis based the products of the first and second set of mental health features to the mental health diagnosis.

As an example, the mental health features extracted from each modality are combined by computing the outer products of the features to obtain the combined data representation before passing through one or more feed forward networks for mental health classification. The inventors have found that the outer product method (i.e. multiplying features from different modalities) is surprisingly effective at diagnosing mental health illness. For example, the inventors showed that the outer product method could incorporate features from two or more of audio, visual, and language data output from a microphone, a camera and a user interface input (or speech to text converter) captured while a patient is speaking in order to accurately screen patients for mental health conditions.

Particularly, the combined data representation is effective in capturing interaction among biomarkers (that is, biomarkers included in the features extracted) from the different modalities. As a result, indications of mental health from the multiple modalities can be effectively combined, which improves accuracy in mental health evaluation.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A is a block diagram of a multi-modal processing system for implementing a multi-modal product fusion model for mental health evaluation, according to an embodiment of the disclosure;

FIG. 1B is a block diagram of a trained multi-modal product fusion model implemented in the multi-modal processing system of FIG. 1A, according to an embodiment of the disclosure;

FIG. 2 is a block diagram of a mental health evaluation system including a plurality of modalities and a trained multi-modal product fusion model, according to an embodiment of the disclosure;

FIG. 3A is a schematic of an architecture of a trained multi-modal product fusion model for mental health evaluation, according to an embodiment of the disclosure;

FIG. 3B is a schematic of an architecture of a trained multi-modal product fusion model for mental health evaluation, according to another embodiment of the disclosure;

FIG. 4 is a schematic of a trained multi-modal product fusion model implemented for mental health evaluation using audio, video, and text modalities, according to an embodiment of the disclosure;

FIG. 5 is a flow chart illustrating an example method for performing mental health evaluation using a trained product fusion model, such as the multi-modal product fusion model at FIG. 3A or FIG. 3B, according to an embodiment of the disclosure; and

FIG. 6 is a flow chart illustrating an example method for training a product fusion model, such as the multi-modal product fusion model at FIG. 3A or FIG. 3B, according to an embodiment of the disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

### Definitions

As used herein, the term “patient” refers to a person or an individual undergoing evaluation for a health condition and/or undergoing medical treatment and/or care.

As used herein, the term “data modality” or “modality data” refers to representative form or format of data that can be processed and that may be output form a particular type of sensor or processed, manipulated, or captured by a sensor in a particular way, and may capture a particular digital representation of a particular aspect of a patient or other target. For example, video data represents one data modality, while audio data represents another data modality. In some examples, three dimensional video represents one data modality, and two dimensional video represents another data modality.

As used herein, the term “sensor” refers to any device for capturing a data modality. The term “sensor type” may refer to different hardware, software, processing, collection, configuration, or other aspects of a sensor that may change the format/type/and digital representation of data output from the sensor. Examples of sensors/types include camera, two dimensional camera, microphone, audio sensors, three dimensional camera, keyboard, user interface, touchscreen, microphone, genetic assays, electrocardiogra sensors, electroencephalography (EEG) sensors, electromyography (EMG) sensors, respiratory sensors, and medical imaging systems including, but not limited to magnetic resonance imaging (MRI) and related modalities such as, functional magnetic resonance imaging (fMRI), T1-weighted MRI, diffusion weighted MRI.

As used herein, the term “mental health” refers to an individual's psychological, emotional, cognitive, or behavioral state or a combination thereof.

As used herein, the term “mental health condition” refers to a disorder affecting the mental health of an individual, and the term “mental health conditions” collectively refers to a wide range of disorders affecting the mental health of an individual. These include, but not limited to clinical depression, anxiety disorder, bipolar disorder, dementia, attention-deficit/hyperactivity disorder, schizophrenia, obsessive compulsive disorder, autism, post-traumatic stress disorder, anhedonia, and anxious distress.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

The present description relates to systems and methods for mental health evaluation using multiple data modalities. In particular, systems and methods are provided for combining multiple data modalities through a multi-modal product fusion model that effectively incorporates indications of mental health from each modality as well as multi-level interactions (e.g., bimodal, trimodal, quadmodal, etc.) between the data modalities.

For instance, in some examples, data modality processing includes a step of producing a product of each of the features of each of the modalities (or particular subsets), in order to output a new set of features that account for complimentary interactions between particular features of particular modalities. Accordingly, this produces product features that will have a higher impact on the classification if both the underlying original features are present or higher. As an example, if a user has a high tone of voice and raises an eyebrow, the combined impact of these features will be captured by a product feature combining a particular voice tone and facial feature that may indicate the likelihood of a particular mental disorder. This is very advantageous for diagnosing mental health disorders, because they are exhibited as a complex constellation of symptoms, that are not captured by systems that process modality by modality.

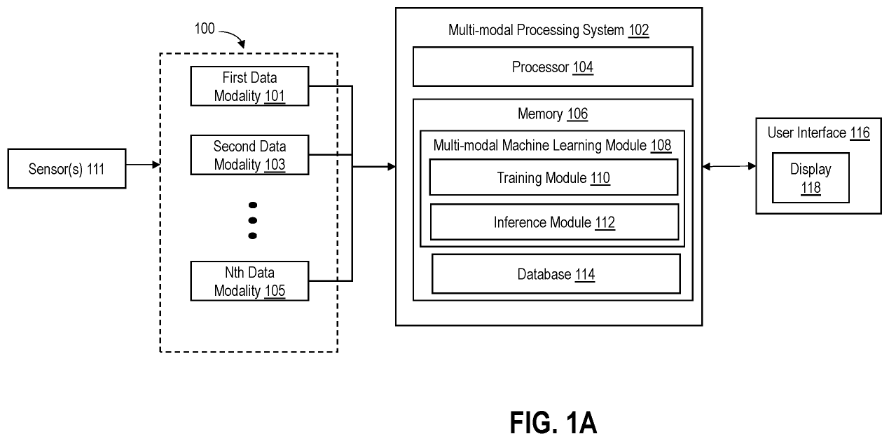

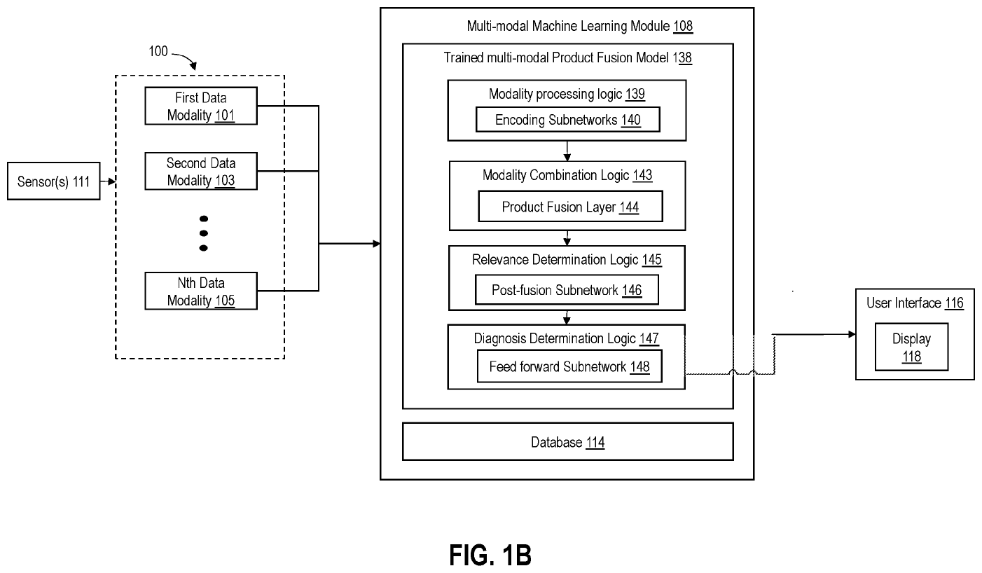

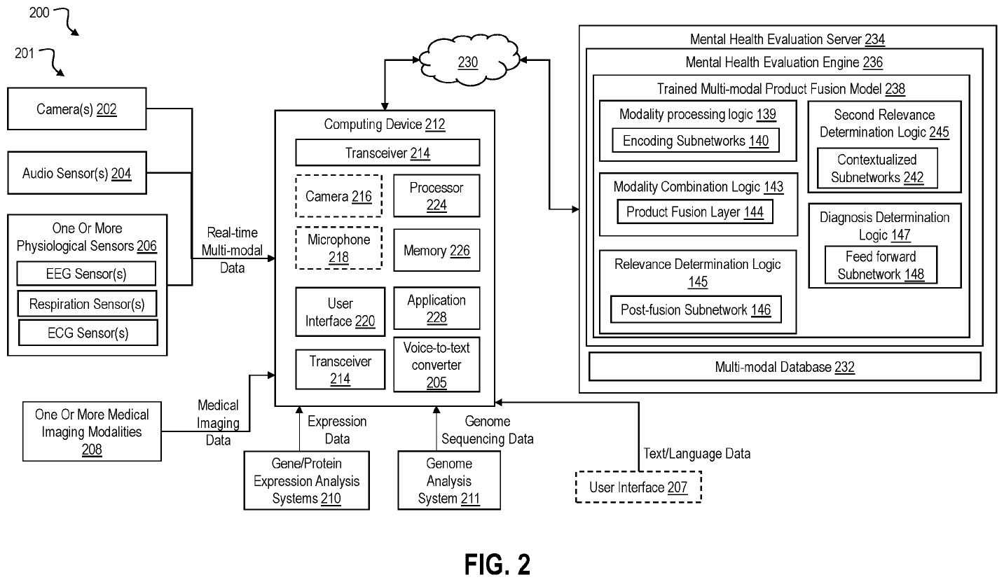

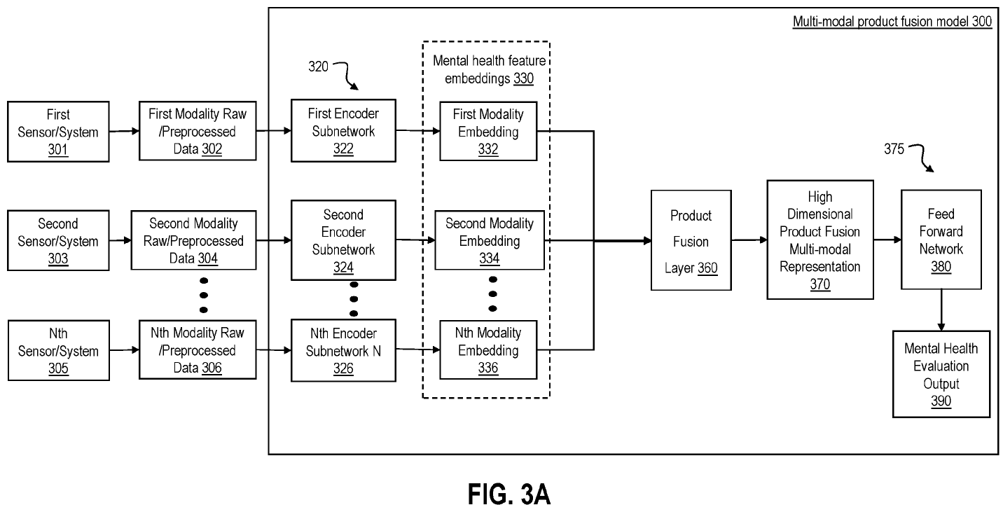

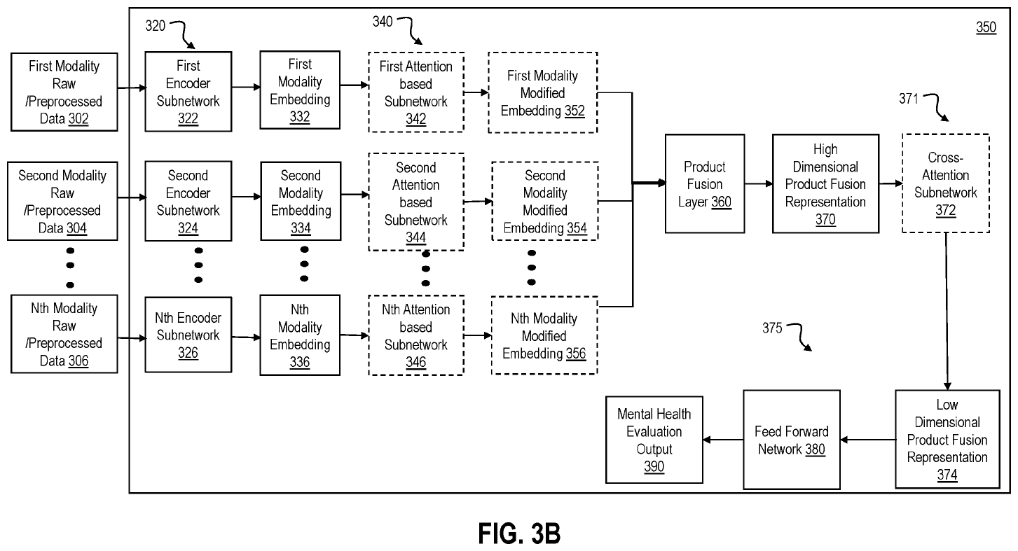

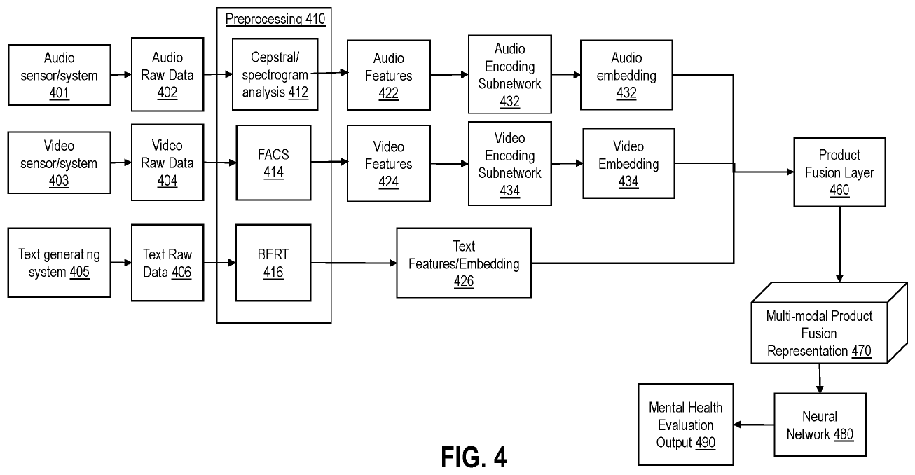

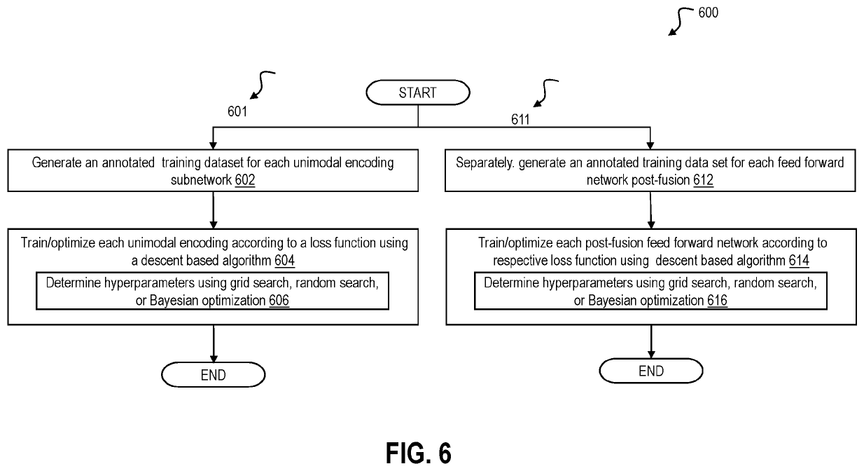

An example multi-modal product fusion model is shown at FIG. 1B and may be implemented in a mental health processing system shown at FIG. 1A. The mental health processing system may be utilized in an example mental health evaluation system illustrated at FIG. 2. An embodiment of a network architecture of the product fusion model is depicted at FIG. 3A, and another embodiment of the network architecture of the product fusion model is depicted at FIG. 3B. In any embodiment, the product fusion model includes a product fusion layer that generates an outer product of mental health features extracted from modality data acquired via one or more sensors and systems. An implementation of the network architecture in FIG. 3A for evaluating mental health using data from audio, video, and text modalities is described at FIG. 4. Example method for evaluating mental health utilizing the product fusion model is discussed with respect to FIG. 6. Further, FIG. 7 shows an example method for training the product fusion model.

The technical advantages of the product fusion model include improved accuracy in mental health evaluation. Particularly, by generating an outer product of the mental health features from a plurality of modalities, interaction between the different modalities is captured in the resulting high dimensional representation, which also includes individual unimodal contributions. For instance, complementary effects between two or more modalities are all captured when using an outer product. When the high dimensional representation is input into one or more classifiers for mental health, the output mental health classification is generated by taking into account the interaction between the different modality data. For example, clinical biomarkers of mental health from an imaging modality combined with evidence of physiological manifestations extracted from one or more of audio, video, and language modalities increases accuracy of mental health evaluation by the product fusion model.

Further, the speed of mental health evaluation and processing is improved by utilizing the product fusion model for mental health evaluation. Specifically, due to the combination of the features from the various modalities generated in the high dimensional representation of the product fusion model, an amount of data required to evaluate mental health symptoms is reduced. Current approaches, whether manual or partly relying on algorithms, are time consuming requiring patient monitoring for a long duration over each assessment session. Even then, the interactions between multiple data modalities are not captured effectively. In contrast, using the product fusion model, mental health evaluation may be performed with shorter monitoring times since the high dimensional representation provides additional information regarding feature interactions among the modalities that allows for faster mental health evaluation. For example, for each data modality, an amount of data acquired may be less, which reduces the duration for data acquisition as well as improves analysis speed. In this way, the product fusion model provides significant improvement in mental health analysis, in terms of accuracy as well as speed.

Further, in some implementations, a self-attention based mechanism is used prior to performing fusion of different data modalities without dimension reduction. As a result, a rich representation of features from each modality is preserved while obtaining context information of features from each data modality. Thus, when fusion is performed, interaction of mental health features from different modalities is captured, which improves accuracy of mental health classification.

### System

FIG. 1A shows a mental health processing system 102 that may be implemented for multi-modal mental health evaluation. In one embodiment, the mental health processing system 102 may be incorporated into a computing device, such as a workstation including a computer at a health care facility. The mental health processing system 102 is communicatively coupled to a plurality of sensors and/or systems generating a plurality of data modalities 100, such as a first data modality 101, a second data modality 103, and so on up to Nth data modality 105, where N is a real number. It will be appreciated that any number of data modalities may be utilized for mental health evaluation. The mental health processing system 102 may receive data from each of the plurality of sensors and/or systems 111. In one example, the mental health processing system 102 may receive data from a storage device which stores the data generated by these modalities. In another embodiment, the mental health processing system 102 may be disposed at a device (e.g., edge device, server, etc.) communicatively coupled to a computing system that may receive data from the plurality of sensors and/or systems, and transmit the plurality of data modalities to the device for further processing. The mental health processing system 102 includes a processor 104, a user interface 114, which may be a user input device, and display 116.

Non-transitory memory 106 may store a multi-modal machine learning module 108. The multi-modal machine learning module 108 may include a multi-modal product fusion model that is trained for evaluating a mental health condition using input from the plurality of modalities 100. Components of the multi-modal product fusion model are shown at FIG. 1B. Accordingly, the multi-modal machine learning module 108 may include instructions for receiving modality data from the plurality of sensors and/or systems, and implementing the multi-modal product fusion model for evaluating a mental health condition of a patient. An example server side implementation of the multi-modal product model is discussed below at FIG. 2. Further, example architectures of the multi-modal product fusion model are described at FIGS. 3A and 3B.

Non-transitory memory 106 may further store training module 110, which includes instructions for training the multi-modal product fusion model stored in the machine learning module 108. Training module 110 may include instructions that, when executed by processor 104, cause mental health processing system 102 train one or more subnetworks in the product fusion model. Example protocols implemented by the training module 110 may include learning techniques such as gradient descent algorithm, such that the product fusion model can be trained and can classify input data that were not used for training. An example method for training the multi-modal product fusion model is discussed below at FIG. 6.

Non-transitory memory 106 also stores an inference module 112 that comprises instructions for testing new data with the trained multi-modal product fusion model. Further, non-transitory memory 106 may store modality data 114 received from the plurality of sensors and/or systems. In some examples, the modality data 114 may include a plurality of training datasets for each of the one or more modalities 100.

Mental health processing system 102 may further include user interface 116. User interface may be a user input device, and may comprise one or more of a touchscreen, a keyboard, a mouse, a trackpad, a motion sensing camera, and other device configured to enable a user to interact with and manipulate data within the processing system 102.

Display 118 may be combined with processor 104, non-transitory memory 106, and/or user interface 116 in a shared enclosure, or may be peripheral display devices and may comprise a monitor, touchscreen, projector, or other display device known in the art, which may enable a user to view modality data, and/or interact with various data stored in non-transitory memory 106.

FIG. 1B depicts the components of the multi-modal product fusion model 138, according to an embodiment. The multi-modal product fusion model is also referred to herein as “product fusion model”. The various components of the product fusion model 138 may be trained separately or jointly.

The product fusion model 138 includes a modality processing logic 139 to process plurality of data modalities from the plurality of sensors 111 to output, for each of the plurality of data modalities, a data representation comprising a set of features. In one example, the modality processing logic 139 includes a set of encoding subnetworks 140, where each encoding subnetwork 140 is a set of instructions for extracting a set of features from each data modality. For example, the modality processing logic 139 and other logic described herein can be embodied in a circuit or the modality processing logic 139 and other logic described herein can be executed by a data processing device such as the multi-modal processing system 102. The subnetworks 140 may be a feed-forward neural network, a convolutional neural network, a long short-term memory network (LSTM), or a transformer or a combination thereof. In some examples, the modality processing logic 139 may further comprise a set of modality preprocessing logic for pre-processing data modalities.

The product fusion model 138 further includes a modality combination logic 143 to process the data representations to output a combined data representation comprising products of each set of features. The modality combination logic 143 includes a product fusion layer 144 including a set of instructions for generating an outer product of the plurality of sets of features from the plurality of data modalities. In particular, the outer product is obtained using all of the data for an entire tensor for each of the plurality of modalities. As a non-limiting example, for a mental health evaluation based on a first modality data, a second modality data, and a third modality data, a combined data representation is obtained using a first tensor, a second tensor, and a third tensor, wherein the first tensor comprises a first data representation of all of the first modality data, the second tensor comprises a second data representation of all of the second modality data, and the third tensor comprises a third data representation of all of the third modality data. Accordingly, the modality combination logic 143 includes a tensor fusion model.

In some examples, the product fusion model 138 includes a relevance determination logic 145 to identify the relevance of each of the products of each set of features to a mental health diagnosis. The relevance determination logic 145 comprises a post-fusion subnetwork 146 which may be a feed-forward neural network, or an attention model. In some examples, a second relevance determination logic may be included before the sets of features are combined by the modality combination logic 143.

Further, the product fusion model 138 includes a diagnosis determination logic 147 to determine a mental health diagnosis based on the relevance of the products to the mental health diagnosis. The mental health diagnosis comprises diagnosis of one or more mental health conditions, the one or more mental health conditions comprising one or more of: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, and a dementia. In some examples, the product fusion model 138 may be utilized to diagnose one or more subtypes of a mental health condition. For example, the product fusion model 138 may be utilized for diagnosis of one or more subtypes of a mental health condition, where the mental health condition is selected from the group consisting of a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, and a dementia.

In one example, the diagnosis determination logic 147 comprises a supervised machine learning model, wherein the supervised machine learning model comprises a random forest, support vector machine, Bayesian Decision List, linear regression, logistic regression, naïve Bayes, linear discriminant analysis, decision tree, k-nearest neighbor, or neural network. In one example, the supervised machine learning model is trained using responses to clinical questionnaires as the outcome label.

Next, FIG. 2 shows a mental health evaluation system 200, according to an embodiment. The mental health evaluation system 200 comprises a plurality of sensors and/or systems 201 that may be utilized to acquire physiological data from a patient for mental health evaluation. Indications of mental health from the plurality of sensors and/or systems 201 are combined via a trained multi-modal product fusion model 238 to provide more accurate and reliable mental health evaluation, as further discussed below.

### Modalities

Following are various examples of modalities and types that may be utilized to implement the system and methods described herein. However, these modalities are only exemplary, and other modalities could be utilized to implement the systems and methods described herein.

### Video and Audio Modalities

The plurality of sensors and/or systems 201 may include at least a camera system comprising one or more cameras 202 and an audio system comprising one or more audio sensors 204. The one or more cameras may include a depth camera, or a two dimensional (2D) camera, or a combination thereof. In one example, the camera system may be utilized to acquire video data. The video data may be used to obtain one or more of movement, posture, facial expression, and/or eye tracking information of the patient.

In one implementation, movement information may include gait and posture information. Accordingly, video data may be used to assess gait, balance, and/or posture of the patient for mental health evaluation, and thus, video data may be used to extract gait, balance, and posture features. In one non-limiting example, a skeletal tracking method may be used to monitor and/or evaluate gait, balance, and/or posture of the patient. The skeletal tracking method includes isolating the patient from the background and identifying one or more skeletal joints (e.g., knees, shoulders, elbows, interphalangeal joints, etc.). Upon identifying a desired number of skeletal joints, gait, balance, and/or posture may be tracked in real-time or near real-time using the skeletal joints. For example, gait, balance, and posture features may be extracted from the video data, and in combination with other features, such as facial expression, gaze, etc., from the video data as discussed further below, may be used to generate a unimodal vector representation of the video data, which is subsequently used for generating a multi-modal representation. As discussed further below, feature extraction from the video data may be performed using a feature extraction subnetwork, which may be a neural network based model (e.g., 1D ResNet, transformer, etc.) or a statistical model (e.g., principal component analysis (PCA)) or other models (e.g., spectrogram for audio data). The feature extraction subnetwork selected may be based on the type of modality (e.g., based on whether the modality is a video modality, audio modality, etc.) and/or the features extracted using the modality data.

In some embodiments, different feature extraction subnetworks may be used for obtaining various sets of features from a single data modality. The output from the different feature extraction subnetworks may be combined to obtain a unimodal representation. For example, while a first feature extraction subnetwork may be used for extracting facial expression features from the video data, a second different feature extraction subnetwork may be used for extracting gait features from the video data. Subsequently, all the features from each modality may be combined, via an encoding subnetwork for example, to obtain a unimodal representation (alternatively referred to herein as unimodal embedding).

Video data may be further used to detect facial expressions for mental health evaluation. In one example, a facial action coding system (FACS) may be employed to detect facial expression from video data acquired with the camera system. The FACS involves identifying presence of one or more action units (AUs) in each frame of a video acquired via the camera system. Each action unit corresponds to a muscle group movement and thus, qualitative parameters of facial expression may be evaluated based on detection of one or more AU in each image frame. The qualitative parameters may correspond to parameters for mental health evaluation, and may include a degree of a facial expression (mildly expressive, expressive, etc.), and a rate of occurrence of the facial expression (intermittent expressions, continuous expressions, erratic expressions etc.). The rate of occurrence of facial expressions may be evaluated utilizing a frequency of the detected AUs in a video sequence. Additionally, or alternatively, a level of appropriateness of the facial expression may be evaluated for mental health assessment. For example, a combination of disparate AUs may indicate an inappropriate expression (e.g., detection of AUs representing happiness and disgust). Further, a level of flatness, wherein no AUs are detected may be taken into account for mental health evaluation. Taken together, video data from the camera system is used to extract facial expression features represented by AUs. The facial expression features may be utilized in combination with the gait, balance, and posture features as well as gaze features for generating a multi-modal representation.

Video data may also be used to evaluate gaze of the patient for mental health assessment. The evaluation of gaze may include a level of focus, a gaze direction, and a duration of gaze. In the evaluation of gaze, movement of eye and pupil behavior (e.g., dilation, constriction) may be tracked using video data. Accordingly, gaze features corresponding to eye movement and pupil behavior may be extracted from the video data and utilized to generate the unimodal vector representation along with gait, balance, posture, and facial expression features discussed above.

In one embodiment, during certain evaluation conditions, such as a remote evaluation condition, a fewer number of features may be extracted from a given modality data, while during some other conditions, such as during a clinical evaluation, a greater number of features may be extracted from the modality data, and considered for mental health evaluation. As a non-limiting example using video data, the fewer number of features may include facial expression, posture, and/or gaze, and the greater number of features may comprise gait and/or balance, in addition to facial expression, posture, and/or gaze. Accordingly, in some examples, the remote evaluation based on the fewer number of features may be used to obtain a preliminary analysis. Subsequently, a second evaluation based on a greater number of features may be performed for confirmation of a mental health condition determined during the preliminary analysis.

The audio system includes one or more audio sensors 204, such as one or more microphones. The audio system is utilized to acquire patient vocal response to one or more queries and tasks. In some examples, audio and video camera systems may be included in a single device, such as a mobile phone, a camcorder, etc. The video recording of the patient response may be used to extract audio and video data. The acquired audio data is then utilized to extract acoustic features indicative of a mental health status of the patient. The acoustic features may include, but not limited to a speech pattern characterized by one or more audio parameters such as tone, pitch, sound intensity, and duration of pause, a deviation from an expected speech pattern for an individual, a fundamental frequency F0 and variation in the fundamental frequency (e.g., jitter, shimmer, etc.), a harmonic to noise ratio measurement, and other acoustic features relevant to mental health diagnosis based on voice pathology. In one example, the acoustic features may be represented by Mel Frequency Cepstral Coefficents (MFCCs) obtained via a cepstral processing of the audio data.

### Physiological Sensor Modalities

While audio and video modalities may be used to characterize behavioral phenotypes, mental health conditions exhibit changes in physiological phenotypes (e.g., ECG activity, respiration, etc.), structural phenotypes (e.g., abnormal brain structure) and associated functional phenotypes (e.g., brain functional activity), and genetic phenotypes (e.g., single nucleotide polymorphism (SNPs), aberrant gene and/or protein expression profile), which may be utilized to obtain a comprehensive and more accurate evaluation of mental health. Therefore, data from physiological sensors, medical imaging devices, and genetic/proteomic/genomic systems may be included in generating a multi-modal representation that is subsequently used to classify mental health condition. Accordingly, the plurality of sensors and/or systems may include one or more physiological sensors 206. The one or more physiological sensors 206 may include Electroencephalography (EEG) sensors, Electromyography (EMG) sensors, Electrocardiogram (ECG) sensors, or respiration sensors, or any combination thereof. Physiological sensor data from each of the one or more physiological sensors may be used to obtain corresponding physiological features representative of mental health. That is, unimodal sensor data representation from each physiological sensor may be obtained according to physiological sensor data from each physiological sensor. Each unimodal sensor representation may be subsequently used to generate a multi-modal representation for mental health evaluation.

### Medical Imaging Modalities

The plurality of modalities 201 may further include one or more medical imaging devices 208. Medical image data from one or more medical imaging devices may be utilized to obtain brain structure and functional information for mental health diagnosis. For example, imaging biomarkers corresponding to different mental health conditions may be extracted using medical image data. Example medical imaging devices include magnetic resonance imaging (MRI) and related modalities such as, functional magnetic resonance imaging (fMRI), T1-weighted MRI, diffusion weighted MRI, etc., positron emission tomography (PET), and computed tomography (CT). It will be appreciated that other medical imaging modalities, in particular, neuroimaging modalities, that provide brain structural and/or functional biomarkers for clinical evaluation of mental health may be used, and is within the scope of the disclosure. Medical image data acquired via one or more medical imaging devices may be used to extract brain structural and functional features (e.g., clinical biomarkers of mental health disease, normal health features, etc.) to generate corresponding unimodal representations. In one example, a plurality of unimodal representations of each medical imaging data modality may be generated, which may be fused to obtain a combined medical image data modality representation. The combined medical image modality representation may be subsequently used to generate multi-modal representation by combining with one or more other modalities (e.g., audio, video, physiological sensors, etc.). In another example, each medical image modality representation (that is, unimodal representation from each medical imaging modality) may be combined with the one or more other modalities without generating the combined medical image modality representation.

### Genetic Modalities

Indications of one or more mental health conditions may be obtained by analyzing one or more of gene expression data, protein expression data, and genetic make-up of a patient. As a non-limiting example, gene expression may be evaluated at a transcript level to determine transcription changes that may indicate one or more mental health conditions. Thus, the plurality of sensors and/or systems 201 may include gene and/or protein expression systems 210. The gene and/or protein expression systems output gene and/or protein expression data that may be used to extract expression changes indicative of mental health conditions. Accordingly, gene and/or protein expression data may be used to generate unimodal representations related to each genetic modality or combined unimodal representations related to multiple genetic modalities. The unimodal or combined unimodal representations may be subsequently used in combination with one or more other modalities discussed above to generate a multi-modal representation for mental health evaluation.

Additionally, or alternatively, genome-wide analysis may be helpful in identifying polymorphisms associated with mental health conditions. Accordingly, the plurality of sensors and/or systems 201 may include a genomic analysis system 211, which may be used to obtain genomic data for mental health analysis. The genomic analysis system 211 may be a genome sequencing system, for example. Genomic data may be used extract genome related features (e.g., features indicative of single nucleotide polymorphisms (SNPs)). The genome related features may be used to generate unimodal genomic representations, which may be combined with gene and/or protein expression features to generate combined genetic representations, which is then used for generating multi-modal representations. Alternatively, the unimodal genomic representations may be combined with one or more other modality representations discussed above to generate multi-modal representations.

### Computing Device(s) for Preprocessing and Implementation of the Product Fusion Model

Mental health evaluation system 200 includes a computing device 212 for receiving a plurality of data modalities acquired via the plurality of sensors and/or systems 201. The computing device 212 may be any suitable computing device, including a computer, laptop, mobile phone, etc. The computing device 212 includes one or more processors 224, one or more memories 226, and a user interface 220 for receiving user input and/or displaying information to a user.

In one implementation, the computing device 212 may be configured as a mobile device and may include an application 228, which represent machine executable instructions in the form of software, firmware, or a combination thereof. The components identified in the application 228 may be part of an operating system of the mobile device or may be an application developed to run using the operating system. In one example, application 228 may be a mobile application. The application 228 may also include web applications, which may mirror the mobile application, e.g., providing the same or similar content as the mobile application. In some implementations, the application 228 may be used to initiate multi-modal data acquisition for mental health evaluation. Further, in some examples, the application 228 may be configured to monitor a quality of data acquired from each modality, and provide indications to a user regarding the quality of data. For example, if audio data quality acquired by a microphone is less than a threshold value (e.g., sound intensity is below a threshold), the application 228 may provide indications to the user to adjust a position of the microphone.

The application 228 may be used for remote mental health evaluation as well as in-clinic mental health evaluation. In one example, the application 228 may include a clinician interface that allows an authenticated clinician to select a desired number of modalities and/or specify modalities from which data may be collected for mental health evaluation. The application 228 may allow the clinician to selectively store multi-modal data, initiate mental health evaluation, and/or view and store results of the mental health evaluation. In some implementations, the application 228 may include a patient interface and may assist a patient in acquiring modality data for mental health evaluation. As a non-limiting example, the patient interface may include options for activating a camera 216 and/or microphone 218 that are communicatively coupled to the computing device and/or integrated within the computing device. The camera 216 and microphone 218 may be used to acquire video and audio data respectively for mental health evaluation.

In one example, memory 226 may include instructions that when executed causes the processor 224 to receive the plurality of data modalities via a transceiver 214 and further, pre-process the plurality of modality data. Pre-processing the plurality of data modalities may include filtering each of plurality of data modalities to remove noise. Depending on the type of modality, different noise reduction techniques may be implemented. In some examples, the plurality of data modalities may be transmitted to mental health evaluation server 234 from the computing device via a communication network 230, and the pre-processing step to remove noise may be performed at server 234. For example, the server 234 may be configured to receive the plurality of data modalities from the computing device 212 via the network 230 and pre-process the plurality of data modalities to reduce noise. The network 230 may be wired, wireless, or various combinations of wired and wireless.

The server 234 may include a mental health evaluation engine 236 for performing mental health condition analysis. In one example, the mental health evaluation engine 236 includes a trained machine learning model, such as a multi-modal product fusion model 238, for performing mental health evaluation using the plurality of noise-reduced (or denoised) data modalities. The multi-modal product fusion model 238 may include several sub-networks and layers for performing mental health evaluation. Example network architectures of the multi-modal product fusion model 238 are described with respect to FIGS. 3A and 3B.

Briefly, the mental health evaluation engine 236 includes one or more modality processing logics 139 comprising one or more encoding subnetworks 140 for generating unimodal feature embeddings using each of the plurality of modality data. In one embodiment, the mental health evaluation engine 236 includes one or more second relevance determination logics 245 comprising one or more contextualized sub-networks 242. Each of the unimodal feature embeddings may be input into corresponding contextualized sub-networks 242 for generating modified unimodal embeddings. The mental health evaluation engine 236 further includes the modality combination logic 143 comprising the product fusion layer 144. The unimodal embeddings or the modified unimodal embeddings are fused at the product fusion layer 144 using a product fusion method to output a multi-modal representation of the plurality of modality data. In one example, each unimodal embeddings or each modified unimodal embeddings may be generated using all of the corresponding modality data without filtering out certain portions of the data and/or removing data from each unimodal embedding. Further, all of each unimodal embeddings are utilized in generating the multi-modal representation or the combined representation. Thus, the multi-modal representation captures all of the modality features as well as all of the modality interactions at various levels. For example, in a mental health evaluation system comprising three data modalities, the multi-modal representation captures unimodal aspects, bimodal interactions, and trimodal interactions. Further, the mental health evaluation engine 236 includes a diagnosis determination logic 147 comprising a feed forward subnetwork 148. The generated multi-modal representation is subsequently input into the feed forward subnetwork 248 to output a mental health classification result or regression result. In some embodiments, the generated multi-modal representation may be input into the relevance determination logic 145 comprising the post-fusion subnetwork 146 for reducing dimensions of the multi-modal representation. The lower-dimensional multi-modal representation is then input into the feed forward subnetwork 148 for classification. Further, the multi-modal product fusion model 238 may be a trained machine learning model. An example training of the multi-modal product fusion model 238 will be described at FIG. 6.

The server 234 may include a multi-modal database 232 for storing the plurality of modality data for each patient. The multi-modal database may also store plurality of training and/or validation datasets for training and/or validating the multi-modal product fusion model for performing mental health evaluation. Further, the mental health evaluation output from the multi-modal product fusion model 238 may be stored at the multi-modal database 232. Additionally, or alternatively, the mental health evaluation output may be transmitted from the server to the computing device, and displayed and/or stored at the computing device 212.

### Multi-Modal Product Fusion Model Architecture

Turning to FIG. 3A, it shows a high-level block diagram of an embodiment 300 of a multi-modal product fusion model, such as the multi-modal product fusion model 238 at FIG. 2. Accordingly, in one example, the multi-modal product fusion model 300 may be implemented by a server, such as server 234 at FIG. 2.

The multi-modal product fusion model 300 (hereinafter referred to as product fusion model 300) has a modular architecture including at least an encoder module 320, a product fusion layer 360, and a mental health inference module 375. The encoder module 320 may be an example of the modality processing logic 143, discussed at FIG. 1B. The encoder module 320 comprises one or more encoder subnetworks 1, 2, etc., and up to N (indicated by 322, 324, and 326 respectively). Each of the one or more encoder subnetworks receives, as input, modality data from at least one of a plurality of sensors and/or systems, such as the plurality of sensors and/or system 201. As shown at FIG. 3A, first modality data 302 acquired from a first sensor 301 is input to the first encoder subnetwork 322, second modality data 304 acquired from a second sensor 303 is input to the second encoder subnetwork 324, and so on up to Nth modality data 306 acquired from a Nth sensor 305 is input to the Nth encoder subnetwork 326.

### Pre-Processing

In one example, one or more of the first modality data 302, the second modality data 304, and up to Nth modality data 306 may be pre-processed before being input to the respective encoder subnetwork. Each modality data may be pre-processed according to the type of data acquired from the modality. For example, audio data acquired from an audio modality (e.g., microphone) may be processed to remove background audio and obtain a dry audio signal of a patient's voice. Video data of the patient acquired from a camera may be preprocessed to obtain a plurality of frames and further, the frames may be processed to focus on the patient or a portion of the patient (e.g., face). Further, when language or text data is preprocessed, noise may be special characters that do not impart useful meaning and thus, noise removal may include removing characters or texts that may interfere with the analysis of text data. Sensor data may be preprocessed by band pass filtering to include sensor data within an upper and lower threshold. In general, the pre-processing of one or more of the first, second, and up to Nth modality data may include one or more of applying one or more modality specific filters to reduce background noise, selecting modality data that has a quality level above a threshold, normalization, and identifying and excluding outlier data, among other modality specific pre-processing. The pre-processing of each modality data may be performed by a computing device, such as computing device 212, before its transmitted to the server for mental health analysis. As a result, less communication bandwidth may be required, which improves an overall processing speed of mental health evaluation. In some examples, the pre-processing may be performed at the server implementing the product fusion model, prior to passing the plurality of modality data through the product fusion model. In some other examples, the product fusion model may be stored locally at the computing device, and thus, the pre-processing as well as the mental health analysis via the product fusion model may be performed at the computing device.

In one embodiment, pre-processing the modality data may include extracting corresponding modality features related to mental health evaluation from the modality data. For example, a rich representation of audio features corresponding to mental health conditions may be generated using audio data from an audio modality (e.g., microphone); a rich representation of video features corresponding to mental health condition may be generated using video data from a video modality (e.g., camera); a rich representation of EEG features corresponding to mental health condition may be generated from EEG data from a EEG sensor; a rich representation of text features associated with mental condition may be generated using text data corresponding to spoken language (or based on user input entered via a user input device); and so on. Feature extraction may be performed using a trained neural network model or any feature extraction method depending on the modality data and/or features extracted from the modality data, where the extracted features include markers for mental health evaluation. An example of feature extraction with respect to a trimodal system for mental evaluation including audio, video, and text data is discussed below with respect to FIG. 4.

### Unimodal Embeddings

Each of the one or more encoding subnetworks in the encoder module 320 generates a unimodal embedding corresponding to its input modality data. In one example, each of the one or more encoding subnetworks receives as input a set of features extracted from the modality data, and generates as output a corresponding modality embedding. As used herein, an “embedding” is a vector of numeric value, having a particular dimensionality. In one embodiment, each of the one or more encoding subnetworks may have a neural network architecture. For example, the one or more encoding subnetworks may be a convolutional neural network (CNN), a recurrent neural network (RNN), a transformer, or any deep neural network or any combination thereof. In one example, a type of architecture of an encoding subnetwork implemented for generating a unimodal embedding may be based on one or more of the modality data, and modality features corresponding to mental health obtained from the modality data. That is, whether the encoding subnetwork is an RNN, a CNN, a transformer network or any neural network may be based on the type of modality data and/or features extracted from the modality data. In some examples, the encoding subnetwork may be a long short term memory (LSTM) network.

### Multi-Modal Representation

Each modality embedding indicates a robust unimodal representation of the mental health features extracted from the corresponding modality data. In order to increase accuracy of mental health diagnosis, the product fusion model 300 includes a product fusion layer 360 that generates a multi-modal representation 370 combining respective unimodal representations of all the modalities. That is, the multi-modal representation 370 is generated by combining all of the modalities and in each modality, all of the unimodal representations are considered for the combination. The multi-modal representation captures unimodal contributions, bimodal interactions, as well as higher order interactions (trimodal, quadmodal, etc.) depending on a number of modalities used for mental health evaluation. The multi-modal representation 370 is generated by computing an outer product of all the unimodal representations from each of the modality data. As a non-limiting example, for a mental health evaluation system acquiring audio modality data, video modality data, text modality data, and EEG modality data, a multi-modal product fusion representation (t) is generated by computing an outer product of unimodal embeddings of all the modalities:

$$t = ( \begin{matrix}w \\ 1\end{matrix} ) ⊗ ( \begin{matrix}x \\ 1\end{matrix} ) ⊗ ( \begin{matrix}y \\ 1\end{matrix} ) ⊗ ( \begin{matrix}z \\ 1\end{matrix} )$$

where w is audio modality embedding, x is video modality embedding, y is text modality embedding, z is EEG modality embedding, and ⊗ indicates the outer product between the embeddings. In this example, the multi-modal product fusion representation 1 models the following: 1. unimodal embeddings w, x, y, and z; 2. bimodal interactions w⊗x, x⊗y, y⊗z, and z⊗x; 3. trimodal interactions w⊗x⊗y, w⊗x⊗z, x⊗y⊗z, and z⊗y⊗w; and 4. quadmodal interactions w⊗x⊗y⊗z. As more modalities are added, the multi modal product fusion representation can be modeled to capture higher order interactions among all modalities. Similarly, when fewer modalities are utilized, the multi-modal product fusion representation may be modeled to capture interactions among all the modalities used.

After generating the multi-modal representation 370, all the dimensions of the multi-modal representation are concatenated into a single multi-modal vector and fed into a mental health inference module 375. The mental health inference module 375 may be an example of diagnosis determination logic 147, discussed at FIG. 1B. The mental health inference module 375 comprises a feed forward neural network 380 and one or more evaluation subnetworks (not shown). The feed forward neural network 380 receives as input the multi-modal vector and outputs a multi-modal embedding that is then passed through the one or more evaluation subnetworks for mental health classification (e.g., binary classification, multi-level classification) and/or regression. The one or more evaluation subnetworks may be one or more neural networks. However, any classifier or regressor may be implemented for mental health classification or regression output.

In this way, the multi-modal product fusion model 300 effectively captures interaction between multiple modalities for mental health evaluation. As such, mental health evaluation using the multi-modal product fusion model 300 takes into account mental health indications obtained from multiple modalities.

In some implementations, during a remote evaluation session, a fewer number of modalities may be available, and hence the fewer number of modalities may be used for a first (or preliminary) mental health evaluation; and during a clinical evaluation session, a greater number of modalities may be used for confirmation of the first (or the preliminary) mental health evaluation. In any case, the product fusion model may automatically adjust weights and biases in the feed forward network 380 for each modality as the number of modalities are increased or decreased.

In some embodiments, mental health analysis may be performed during a plurality of sessions, and an aggregated score from the plurality of sessions may be utilized to confirm a mental health condition.

FIG. 3B shows a high-level block diagram of another embodiment 350 of the multi-modal product fusion model. In this embodiment, in addition to the encoder module 320, the product fusion later 360, and the mental health evaluation module 375, one or more attention-based modules may be included in the multi-modal product fusion model.

In one implementation, a post-fusion module 371 may be added downstream of the product fusion layer 360 and upstream of the mental health evaluation module 357. The post-fusion module 371 may receive the multi-modal product fusion representation 370 (that is, the outer product of all unimodal embeddings) as input, and generate a lower dimensional product fusion representation 374. The post-fusion module 371 may be an example of relevance determination logic 14, discussed at FIG. 1B.

In one example, the post-fusion module 371 may be implemented by a cross-attention mechanism. For example, given a number of input streams (m), where the input streams can be individual modalities, or outputs of tensor product. Further, when d1, d2, and so on upto dm are the dimensions of these input streams. These streams are reshaped to a common dimension d via linear transformation and these are concatenated together to form matrix G=[G1, G2, . . . Gm]∈Rd×m where Gi is the i^{th} stream. d is determined by hyperparameter tuning. The cross-attention fusion is performed as follows:

$$P = tanh ( W \cdot G )$$

$$α = softmax ( w \cdot P )$$

$$F = G α T$$

Where, αT∈Rm is the fusion weight for the m streams and F∈Rd is the fused embedding going to the feed-forward layer and W and w are trained through back-propagation.

In another example, any dimensionality reduction method may be used for implementing the post-fusion module 371. Since different degrees of interactions (unimodal contributions, bimodal interactions, trimodal interactions, etc.) between the modalities are already captured in the multi-modal product fusion representation 370, any dimensionality reduction method may be used to reduce a number of input variables for the subsequent feed forward network 380, and select features that are important for mental health evaluation. That is, since all the interactions are already captured in the product fusion representation 370, using any dimension reducing mechanism the inter-modal and intra-modal interactions can still be preserved. The dimensionality reduction method may be an attention based mechanism, or other known supervised dimension reduction models. As an example, it may be ambiguous to pinpoint the mental state based on one modality for example a neutral text modality. However, in combination with other modalities (e.g. a flat tone and/or a frown), the neutral text modality may be a more significant indicator. The multi-modal interaction is modeled explicitly through the tensor product operation where any combination of features in any modality is allowed to interact. The resulting dimension of this fusion is often very large and may result in overfitting while training the feed-forward neural network. Hence, in one example, a drop-out or implicit feature selection through attention may be utilized before putting the product fusion representation through the feed-forward neural network 380.

In another implementation, a pre-fusion module 340 may be included between the encoder module 320 and the product fusion layer 360. The pre-fusion module 340 may include a plurality of attention based subnetworks including a first attention based subnetwork 342, a second attention based network 344, and so on up to a Nth attention based subnetwork 346. In one example, each of the plurality of attention based subnetworks may implement a multihead self-attention based mechanism to generate contextualized unimodal representations that are modified embeddings having context information. In particular, the modified embeddings are generated without undergoing dimension reduction in order to preserve rich representation of the embedding. This, improves model performance. In particular, generating modified embeddings using attention based mechanisms without reducing dimensions before fusion improves model performance for mental health classification, as it preserves features extracted from each data modality. Thus, when the unimodal (modified) embeddings are combined by product fusion, various feature interaction combinations are generated. As a result, accuracy of mental health classification is improved. Accordingly, the first attention based subnetwork 342 receives the first modality embedding 332 as input and outputs a first modality modified embedding 352, the second attention based subnetwork 344 receives the second modality embedding 334 as input and outputs a second modality modified embedding 354, and so on until Nth attention based subnetwork 346 receives the Nth modality embedding 356 and outputs a Nth modality modified embedding. Each modified modality embedding includes context information relevant to each modality. In this way, by passing each modality embedding through a multi-head self-attention mechanism, contextualized unimodal representations (that is, modified embeddings) may be generated. In one non-limiting example, considering unimodal embeddings of m modalities with d dimensions, where the m modalities have not interacted with each other at this point. The unimodal embeddings are more predictive if those are contextualized. That is, the unimodal embeddings are generated while taking interactions among multiple modalities into account. This is done through self-attention. At the end of this step, the result is still m embeddings with d dimensions each, but now these embeddings are contextualized. In some examples, there may be multiple contexts that needs to be taken into account which one self-attention procedure may not accommodate. In such examples, the self-attention procedure may be performed in parallel multiple times, and as such, referred to as multihead attention.

### Example Trimodal Mental Health Evaluation

FIG. 4 shows an example of multi-modal mental health evaluation by employing a multi-modal product fusion model, such as the multi-modal product fusion model 300, with data from audio, video, and text modalities.

### Data Acquisition

In order to assess mental health condition, a patient is provided with a plurality of tasks and/or plurality of queries, and the patient response is evaluated using multiple data modalities. The plurality of tasks may include, but not limited to, reading a passage, performing specified actions (e.g., walking, input information using a user interface of a computing system, etc.), responding to open ended questions, among other tasks. The patient response to the plurality of tasks and/or the plurality of queries is captured using an audio sensor 401 (e.g., microphone), a video system 403 (e.g. camera), and a text generating system 405 (e.g., user text input via the user interface, speech to text input by converting spoken language to text). The mental health assessment using audio, video, and text modalities may be performed remotely with guidance, queries, and/or tasks provided via a mental health assessment application software, such as application 248 at FIG. 2, or from a health care provider remotely communicating with the patient, or a combination thereof. In some examples, the mental health assessment may be performed in-clinic, wherein a health care provider may instruct the patient to perform the plurality of tasks and/or ask the plurality of questions. Additionally, or alternatively, the mental health assessment application may also be utilized for in-clinic evaluation. In any example, two or more modalities may be used to evaluate patient response for diagnosing a mental health condition.

### Pre-Processing/Feature Extraction

Audio data 402 acquired from the audio sensor 401, video data 404 acquired from the video system 403, and text data 406 from the text generating system 405 are pre-processed in a modality-specific manner. In one example, all of the audio data is processed to output an audio data representation comprising an audio feature set; all of the video data is processed to output a video data representation comprising a video feature set; all of the text data is processed to output a text data representation comprising a text feature set.

The audio data 402 is preprocessed to extract audio features 422. Prior to extracting features, one or more signal processing techniques, such as filtering (e.g. Weiner filter), trimming, etc, may be implemented to reduce and/or remove background noise and thereby, improve an overall quality of the audio signal. Next, audio features 422 are extracted from the denoised audio data using one or more of a Cepstral analysis and a Spectrogram analysis 412. The audio features 422 include Mel-Frequency Cepstral Coefficients (MFCC) obtained from a plurality of mel-spectrograms of a plurality of audio frames of the audio data. In some examples, spectrograms and/or Mel-spectrograms may be used as audio features 422. Additionally, audio features 422 comprise features related to mental health evaluation, including voice quality features (e.g., jitter, shimmer, fundamental frequency F0, deviation from fundamental frequency F0), loudness, pitch, formants, among other features for clinical evaluation.

The video data 404 is preprocessed to extract video features 424. Similar to audio data, one or more image processing techniques may be applied to video data to remove unwanted background or noise prior to feature extraction. Video feature extraction is performed according to a Facial Action Coding System (FACS) that captures facial muscle changes, and the video features include a plurality of action units (AU) corresponding to facial expression in each of a plurality of video frames. In addition to AUs relating facial expressions, one or more other video features may be extracted which facilitate in mental health analysis. The one or more other video features may include posture features, movement features (e.g., gait, balance, etc.), eye tracking features, may also be obtained from video data 404. For example, while a patient's facial expression is monitored using action units, shoulder joint position and head position may be simultaneously obtained by passing the same set of video frames through a model for posture detection. In some examples, the AUs may also capture posture information. In another example, a patient may be provided with a balancing task, which may include walking. Accordingly, a skeletal tracking model that identifies and tracks joints and connection between the joints may be applied to the video data to extract balance features and gait features.

The text data 406 is processed to generate text features 426 according to a Bidirectional Encoder Representations from Transformers (BERT) model 416. BERT has a bidirectional neural network architecture, and outputs contextual word embeddings for each word in the text data 406. Accordingly, the text features 426 comprise contextualized word embeddings, which are directly utilized for product fusion with audio and video embeddings at the subsequent product fusion layer.

### Unimodal Audio, Video, and Text Embeddings

Audio features 422 and video features 424 are input into respective audio and video encoding subnetworks 432 and 434 to obtain audio embedding 432 and video embedding 434 respectively. The audio and video encoding subnetworks 432 and 434 may have a neural network architecture. In one example, each of the audio and video subnetworks may be modelled according to a deep network, such as ResNet, or any other suitable convolutional backbone, which may process the input audio and video features to generate corresponding audio and video embeddings 432 and 434.

In one embodiment, the audio and video embeddings may be further modified using a multihead self-attention mechanism to contextualize the audio and video embeddings.

### Multi-Modal Product Fusion Representation

The audio, video, and text embeddings are fused by computing an outer product of the audio, video, and text embeddings at a product fusion layer 460. The outer product of the audio, video, and text embeddings is high-dimensional and captures unimodal contributions as well as bimodal and trimodal interactions. Further, at the product fusion layer 460, all the dimensions of the outer product are concatenated into a single vector, which is fed into a feed forward network, which may be any neural network, such as a convoluted neural network (CNN), to obtain a multi-modal product fusion representation 470.

### Application Layer

The multi-modal product fusion representation 470 can be utilized in a variety of applications, including supervised classification, supervised regression, supervised clustering, etc., Accordingly, the multi-modal product fusion representation 470 is fed into one or more neural networks 480. The neural networks 480 may each be trained to classify one or more mental health conditions or output a regression result for a mental health condition.

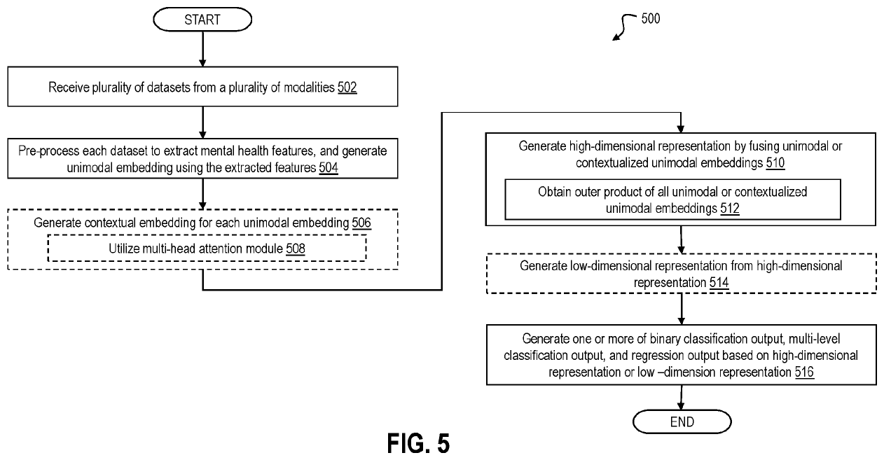

Turning to FIG. 5, it shows a flow chart illustrating a high-level method 500 for evaluating a mental health condition of a patient based on multi-modal data from a plurality of modalities. The method 500 may be executed by a processor, such as processor 224 or one or more processors of mental health evaluation server 234 or a combination thereof. The processor executing the method 500 includes a trained multi-modal product fusion model, such as model 300 at FIG. 3A and/or model 350 at FIG. 3B. As discussed above, the trained multi-modal product fusion model is trained to classify one or more mental health conditions, including but not limited to depression, anxious depression, and anhedonic conditions, or output a regression result pertaining to the one or more health conditions.

In one example, the method 500 may be initiated responsive to a user (e.g., a clinician, a patient, a caregiver, etc.) initiating mental health analysis. For example, the user may initiate mental health analysis via an application, such as app 228. In another example, the user may initiate mental health data acquisition; however, the data may be stored and the evaluation of mental health condition may be performed at a later time. For example, mental health analysis may be initiated when data from a desired number and/or desired types of modalities (e.g., audio, video, text, and imaging) are available for analysis. The method 500 will be described below with respect to FIGS. 2, 3A and 3B; however, it will be appreciated that the method 500 may be implemented by other similar systems.

At 502, the method 500 includes receiving a plurality of datasets from a plurality of sensors and/or systems. The plurality of sensors and/or systems include two or more of the sensors and/or systems 201 described at FIG. 2. For example, the plurality of sensors and/or systems may include two or more of audio, video, text, physiological sensor, medical imaging, gene expression, protein expression, and genomic modalities, such as camera system 202, audio sensors 204, user interface 207, voice to text converter 205, one or more physiological sensors 206, one or more medical imaging modalities 208, gene and/or protein expression system 210, and genomic modality 211. Other systems, such as metabolomic profiling/analytic systems including nuclear magnetic resonance spectrometry (NMR), gas chromatography mass spectrometry (GC-MS) and liquid chromatography mass spectrometry (LC-MS) may also be integrated into the mental health evaluation system, and as such, metabolic data generated from one or more metabolic profiling/analytic systems may be utilized for mental health evaluation. As a non-limiting example, in a trimodal system, a patient response may be evaluated using a video recording, and patient input via the user interface. As such, video data, and audio data from the recording, and text data according to text converted from spoken language via the speech to text converter and/or patient text input via the user interface may be transmitted to the processer implementing the trained multi-modal product fusion model. In some examples, modality data may be processed in real time using the product fusion model, and real-time or near real-time mental health evaluation by implementing the product fusion model is also within the scope of the disclosure.

Next, at 504, the method 500 includes pre-processing each of the plurality of datasets to extract mental health features from each dataset, and generating unimodal embeddings from each dataset based on the extracted mental health features. In one example, pre-processing each of the plurality of datasets includes reducing and/or removing noise from each raw dataset. For example, a signal processing method, such as band-pass filtering may be used to reduce or remove noise from a dataset. Further, the type of signal processing used may be based on the type of dataset. Pre-processing each dataset further includes passing the noise-reduced/denoised dataset or the raw dataset through a trained subnetwork, such as a trained neural network, for extracting a plurality of mental health features from each dataset. Any other feature extraction method that is not based on neural networks may be also used.

Continuing with the trimodal example above, a plurality of frames of the video data may be passed through a trained neural network model comprising a trained convoluted neural network for segmenting, identifying and extracting a plurality of action units according to FACS. Further, audio data may be processed to generate a cepstral representation of the audio data and a plurality of MFCC may be derived from the cepstral representation, and text data may be processed according to pre-trained or fine-tuned BERT model to obtain one or more sequences of vectors. In some examples, one or more datasets may be preprocessed using statistical methods, such as principal component analysis (PCA), for feature extraction. As a non-limiting example, EEG data may be preprocessed to extract a plurality of EEG features pertaining to mental health evaluation.

Upon extracting mental health features from each dataset, the features from each dataset may be passed through a corresponding trained encoding subnetwork to generate unimodal embeddings for each dataset. For example, a set of mental features extracted from a dataset may be input in to a trained encoding neural network to generate unimodal embeddings, which are vector representations of the input features for a given modality. In this way, unimodal embeddings for each modality used for mental health evaluation may be generated.

Turning to the trimodal example, a trained video encoding subnetwork, such as a trained 1D RESNET, may receive the extracted audio features (e.g., MFCC and/or spectrograms) as input and generate video embeddings as output. Similarly, a trained audio encoding subnetwork, such as a second trained 1D RESNET, may receive the extracted video features (e.g., Action units) as input and generate audio embeddings as output. With regard to text data, as the output of the pre-trained or fine-tuned BERT model is a vector sequence, the output itself is the text embedding.

Next, in one embodiment, method 500 proceeds to 506, at which step the method 500 includes generating contextual embedding for one or more unimodal embeddings. In one example, an attention based mechanism, such as a multi head self-attention mechanism may be used to generate contextual embedding from one or more unimodal embeddings. In some examples, only some unimodal embeddings may be modified to generate contextual embeddings while remaining unimodal embeddings may not be modified and used without contextual information to generate multi-modal representation. In some other examples, all the unimodal embeddings may be modified to obtain respective contextual embeddings.

In another embodiment, the method 500 may not generate contextual embeddings, and may proceed to step 510 from 506. At 510, the method 500 includes generating a high-dimensional representation of all modalities by fusing the unimodal embeddings or the contextualized embeddings or a combination of unimodal and contextualized embeddings. The high-dimensional representation may be obtained by generating an outer product of all the embeddings. For example, in a mental health evaluation system comprising N number of modalities, where N is a real number greater than or equal to two, N number of unimodal embeddings are generated, and one multi-modal high dimensional representation is obtained by generating an outer product of the N number of unimodal embeddings. Details of generating the outer product are discussed above with respect to the product fusion layer 360 at FIG. 3A.

Continuing with the trimodal example above, the audio, video, and the text embeddings may be fused by generating an outer product of all of the audio embeddings, all of the video embeddings, and all of the text embeddings. Said another way, a trimodal product fusion representation may be obtained by computing an outer product of the audio, video, and text vectors. If the audio vector is represented by a, the video vector is represented by v, and the text vector is represented by t, trimodal product fusion representation tp is obtained by:

$$t p = ( \begin{matrix}a \\ 1\end{matrix} ) ⊗ ( \begin{matrix}v \\ 1\end{matrix} ) ⊗ ( \begin{matrix}t \\ 1\end{matrix} )$$

As discussed above with respect to FIGS. 3A and 3B, by obtaining the outer product of the unimodal tensors, in addition to contribution of each modality, higher level interactions (e.g., bimodal and trimodal interactions in case of trimodal system discussed herein) are included in the high dimensional representation.

Next, in one embodiment, upon obtaining the high dimensional representation at 510, the method 500 proceeds to 514 to generate a low dimensional representation. In one example, a cross-attention mechanism may be utilized to generate the low dimensional representation. In other examples, any other dimensionality reduction method may be implemented. In particular, since the interactions between the different modalities are captured in the high dimensional representation, any dimensionality reduction mechanism may be used and the interacting features for mental health determination would still be preserved. The dimensionality reduction mechanisms may include a feed-forward neural network, a convolutional neural network, a long short-term memory network (LSTM), or a transformer. Upon obtaining the low dimensional representation, the method 500 proceeds to 516.

In another embodiment, the method 500 may proceed from step 510 to 516 to generate one or more of mental health evaluation outputs. In particular, at 516, generating the one or more mental health evaluation outputs includes inputting the high dimensional representation (or the low dimensional representation if step 514 is performed) into a trained mental health inference module, such as the mental health inference module 375 at FIGS. 3A and 3B. The trained mental health reference module may include one or more feed forward networks. For example, a first feed forward network trained by a supervised classification method may be used to output a binary classification result (e.g., depressed or not depressed). A second feed forward network may be trained by a supervised classification method to output a multi-class classification result (e.g., different levels of depression). A third feed forward network may be trained by a supervised regression method to output a regression result, which may be further used for multiclass or binary classification.

In one embodiment, depending on a number of modalities, the method 500 may determine whether to reduce the dimensions of the high dimensional representation. For example, if the number of modalities is greater than a threshold number, the dimension reduction mechanism may be implemented to generate the low dimension representation prior to inputting into the mental health inference module. However, if the number of modalities is at or less than the threshold number, the high dimensional representation may be directly input into the mental health inference module to obtain one or more mental health evaluation outputs.

### Training

FIG. 6 shows a flowchart illustrating a high-level method 600 for training a product fusion model for mental health evaluation, such as product fusion model 300 at FIG. 3A. The method 600 may be executed by a processor 104 according to instructions stored in non-transitory memory 106. In general, training of one or more encoder subnetworks, such as the one or more encoder subnetworks of encoder module 320 at FIG. 3A, and training of one or more feed forward networks that are used post-fusion (that is, using multi-modal representation as input) may be performed jointly or separately. The method 600 shows example training method when performed separately.

Whether performed separately or jointly, any descent based algorithm may be used for training purposes. A loss function used for training may be based on the application for the feed forward network. For example, for a classification application, loss functions may include cross-entropy loss, hinge embedding loss, or KL divergence loss. For a regression application, Mean Square Error, Mean Absolute Error, or Root Mean Square Error may be used. Further, under both joint and separate training situations, hyperparameters to help guide learning may be determined using a grid search, random search, or Bayesian optimization algorithms.

Branch 601 shows high-level steps for training unimodal subnetworks that are used to generate unimodal embeddings (or unimodal representations) before generating multi-modal representation combining the unimodal embeddings; and branch 611 shows high-level steps for training one or more feed forward networks that are used for mental health classification with the multi-modal representation.

Training unimodal subnetworks includes at 602, generating a plurality of annotated training datasets for each data modality. In one example, for a trimodal mental health evaluation using audio, video, and text data, the training dataset may be based on a set of video recordings acquired via a device. Using the video recordings, trimodal data comprising audio data (for evaluating vocal expressions, modulations, changes, etc.), video data (for evaluating facial expressions, body language etc.), and text data (for evaluating linguistic response to one or more questions) may be extracted. For example, video recordings of a threshold duration (e.g., 1 minute, 2 minutes, 3 minutes, 4 minutes, 5 minutes, 6 minutes, 7 minutes, 8 minutes, 9 minutes, 10 minutes, or more than 10 minutes) from each of a plurality of subjects may be acquired via a camera and microphone of a computing device or via a software application running on the computing device using the camera and the microphone. Further, from each of the video recordings, audio, video, and text datasets may be extracted and labelled according to one or more clinical scales for mental health conditions. The one or more clinical scales may include one or more of a clinical scale for a depressive disorder, a clinical scale for an anxiety disorder, and a clinical scale for anhedonia. Depending on the mental health conditions analyzed the corresponding clinical scales may be used. The labelled audio data, the labelled video data, and the labelled text data may be used for training the corresponding subnetworks in multimodal product fusion model. An example dataset used for training an example multimodal product fusion model for assessing one or more of a depressive disorder, anxiety disorder, and an anhedonic condition is described below under the experimental data section.

Next, at 604, each unimodal subnetwork is trained using its corresponding training dataset by a descent based algorithm to minimize loss function. For example, after each pass with the training dataset, weights and bias at each layer of the subnetwork may be adjusted by back propagation according to a descent based algorithm so as to minimize the loss function. Hyperparameters used for training may include a learning rate, batch size, a number of epochs, and activation function values, and may be determined using any of grid search, random search, or Bayesian search as indicated at 606. Training the one or more feed forward networks may be performed as indicated at steps 612, 614, and 616, using a post-fusion annotated training dataset. The training is based on the multimodal data. For example, initially if there are n participants. For each participant, m modalities of data and a score/label (e.g., depending on whether regression/classification is performed) are obtained. After the fusion step (e.g., after product fusion layer 360 or 460), each participant has a m dimensional representation and we have a n×m data matrix and n scores/labels. The feedforward network takes n×m as input and performs regression/classification using the n scores/labels. The fusion representations would be trained jointly with this feed-forward network.

When performing joint training, the back propagation is performed with respect to the entire network i.e. gradients are propagated backward starting from the feedforward layer back to the individual modality subnets to optimize the weights of the modality subnets as well as the feed-forward network simultaneously.

In one embodiment, a device, comprises a first modality processing logic to process a first data modality from a first type of sensor to output a first data representation comprising a first set of features; a second modality processing logic to process a second data modality from a second type of sensor to output a second data representation comprising a second set of features; modality combination logic to process the first and second data representations to output a combined data representation comprising products of the first and second set of features; relevance determination logic to identify the relevance of each of the products of the first and second features to a mental health diagnosis; and diagnosis determination logic to determine a mental health diagnosis based on the relevance of the products of the first and second set of features to the mental health diagnosis. In a first example of the device, the first and second sensor type each comprise one of: a camera, a microphone, a MRI scanner, a user interface, a keyboard, an EEG detector, or a plate reader. In a second example, which optionally includes the first example, the first and second modality processing logic each further comprise a first and second modality preprocessing logic. In a third example, which optionally includes one or more of the first and second examples, the first and second modality preprocessing logic comprises a feature dimensionality reduction model. In a fourth example, which optionally includes one or more of the first through third examples, the first and second modality processing logic comprises at least one of: a feed-forward neural network, a convolutional neural network, a long short-term memory network (LSTM), or a transformer. In a fifth example, which optionally includes one or more of the first through fourth examples, the modality combination logic comprises a tensor fusion model, the tensor fusion model configured to generate the combined data representation based on an outer product of all of the first set of features and all of the second set of features. In a sixth example, which optionally includes one or more of the first through fifth examples, the relevance determination logic comprises at least one of a feed-forward neural network, or an attention model. In a seventh example, which optionally includes one or more of the first through sixth examples, diagnosis determination logic comprises a supervised machine learning model. In an eighth example, which optionally includes one or more of the first through seventh examples, the supervised machine learning model comprises a random forest, support vector machine, Bayesian Decision List, linear regression, logistic regression, naïve Bayes, linear discriminant analysis, decision tree, k-nearest neighbor, or neural network. In a ninth example, which optionally includes one or more of the first through eighth examples, the supervised machine learning model is trained using responses to clinical questionnaires as the outcome label. In a tenth example, which optionally includes one or more of the first through eleventh examples, the first and second modality processing logic is trained separately from the relevance determination logic. In an eleventh example, which optionally includes one or more of the first through tenth examples, the first and second modality processing logic is trained jointly with the relevance determination logic. In a twelfth example, which optionally includes one or more of the first through eleventh examples, the camera is a three dimensional camera. In a thirteenth example, which optionally includes one or more of the first through twelfth examples, the mental health diagnosis comprises at least one of: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, or a dementia. In a fourteenth example, which optionally includes one or more of the first through thirteenth examples, the mental health diagnosis comprises a quantitative assessment of a severity of the mental health disorder.

In another embodiment, a device comprises a first modality processing logic to process data output from a first type of sensor to output a first set of features; a second modality processing logic to process data output from a second type of sensor to output a second set of features; a product determination logic to determine a product of the first and second set of features; a diagnostic relevance interaction logic to identify a relevance of each of the products of the first and second set of features to a mental health diagnosis; and a diagnosis determination logic to determine a mental health diagnosis based on the diagnostic relevance of each of the products of the first and second set of features. In one example of the device, the device further comprises a third modality processing logic to process data output from a third type of sensor to output a third set of features. In a second example, which optionally includes the first example, the product of the first and second set of features comprises the product of the first, second, and third set of features. In a third example, which optionally includes one or more of the first and the second examples, the relevance of the first and second set of features comprises the relevance of the first, second, and third set of features. In a fourth example, which optionally includes one or more of the first through third examples, the diagnostic relevance of each of the products of the first and second set of feature further comprises the diagnostic relevance of each of the products of the first, second, and third set of features. In a fifth example, which optionally includes one or more of the first through fourth examples, the first type of sensor comprises a camera, the second type of sensor comprises a microphone, and the third type of sensor comprises a user interface configured to receive textual user input. In a sixth example, which optionally includes one or more of the first through fifth examples, the first set of features comprises facial features, the second set of features comprises voice features, and the third set of features comprises textual features.

In another embodiment, a computing device comprises: a memory containing machine readable medium comprising machine executable code having stored thereon instructions; and a control system coupled to the memory comprising one or more processors, the control system configured to execute the machine executable code to cause the control system to: receive a first set of data comprising a first data modality output from a first type of sensor; receive a second set of data comprising a second data modality from a second type of sensor; receive a third set of data comprising a third data modality output from a third type of sensor; process the first set of data with a first model to output a first data representation comprising a first feature set; process the second set of data with a second model to output a second data representation comprising a second feature set; process the third set of data with a third model to output a third data representation comprising a third feature set; and process the first, the second, and the third data representations with a product model to output a set of combination features, wherein each of the set of combination features comprising products of the first, second, and third feature set; and process the set of combination features using a fourth model to output a combined data representation. In a first example of the computing device, the first, second, and third type of sensor each comprise one of: a camera, a microphone, a MRI scanner, a user interface, a keyboard, an EEG detector, or a plate reader. In a second example, which optionally includes the first example, the first data modality comprises image data, video data, three dimensional video data, audio data, MRI data, text strings, EEG data, gene expression data, ELISA data, or PCR data. In a third example, which optionally includes one or more of the first and the second examples, the camera comprises a three dimensional camera. In a fourth example, which optionally includes one or more of the first through third examples, the product model is a tensor fusion model. In a fifth example, which optionally includes one or more of the first through fourth examples, the mental health classification comprises: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, or a dementia. In a sixth example, which optionally includes one or more of the first through fifth examples, process the set of combination features using a fourth model further comprises first processing the set of combination features using an attention model. In a seventh example, which optionally includes one or more of the first through sixth examples, the first, second, and third data representation comprise feature vectors. In an eighth example, which optionally includes one or more of the first through seventh examples, the first, second, and third data modality each comprise a unique data format. In a ninth example, which optionally includes one or more of the first through eighth examples, the first data representation comprises an output from a convolution neural network, long short-term memory network, transformer, or a feed forward neural network. In a tenth example, which optionally includes one or more of the first through ninth examples, the first model comprises a feed-forward neural network, a convolutional neural network, a long short-term memory network (LSTM), or a transformer. In an eleventh example, which optionally includes one or more of the first through tenth examples, the fourth model comprises a feed-forward neural network. In a twelfth example, which optionally includes one or more of the first through eleventh examples, the control system is further configured to execute the machine executable code to cause the control system to process the combined data representation with a supervised machine learning model to output a mental health classification of a patient. In a twelfth example, which optionally includes one or more of the first through eleventh examples, the first, second and third models are trained separately from the fourth model. In a thirteenth example, which optionally includes one or more of the first through twelfth examples, the first, second, third and fourth models are trained jointly. In a fourteenth example, which optionally includes one or more of the first through thirteenth examples, the supervised machine learning model comprises a random forest, support vector machine, Bayesian Decision List, linear regression, logistic regression, naïve Bayes, linear discriminant analysis, decision tree, k-nearest neighbor, or neural network.

In another embodiment, a computing device comprises a memory containing machine readable medium comprising machine executable code having stored thereon instructions; and a control system coupled to the memory comprising one or more processors, the control system configured to execute the machine executable code to cause the control system to: receive a first set of data comprising a first data modality output from a first type of sensor; receive a second set of data comprising a second data modality from a second type of sensor; receive a third set of data comprising a third data modality output from a third type of sensor; process all of the first set of data with a first model to output a first data representation comprising a first feature set; process all of the second set of data with a second model to output a second data representation comprising a second feature set; process all of the third set of data with a third model to output a third data representation comprising a third feature set; and process all of the first, all of the second, and all of the third data representations with a product model to output a set of combination features, wherein each of the set of combination features comprising products of the first, second, and third feature set; and process the set of combination features using a fourth model to output a combined data representation.

In another embodiment, a device comprises: a modality processing logic to process data output from at least three types of sensors to output a set of data representations for each of the at least three types of sensors, wherein each of the set of data representations comprises a vector comprising a set of features; modality combination logic to process the set of data representations to output a combined data representation comprising an outer product of the set of data representations; relevance determination logic to identify the relevance of each of the outer product to a mental health diagnosis; and diagnosis determination logic to determine a mental health diagnosis based on the relevance of the outer product to the mental health diagnosis. In a first example of the device, the at least three types of sensors each comprise at least one of: a camera, a microphone, a MRI scanner, a user interface, a keyboard, an EEG detector, or a plate reader. In a second example, which optionally includes the first example, the modality processing logic further comprises a preprocessing logic. In a third example, which optionally includes one or more of the first and the second examples, the preprocessing logic comprises a feature dimensionality reduction model. In a fourth example, which optionally includes one or more of the first through third examples, the modality processing logic comprises at least one of: a feed-forward neural network, a convolutional neural network, a long short-term memory network (LSTM), or a transformer. In a fifth example, which optionally includes one or more of the first through fourth examples, the modality combination logic comprises a tensor fusion model. In a sixth example, which optionally includes one or more of the first through fifth examples, the relevance determination logic comprises at least one of a feed-forward neural network, or an attention model. In a seventh example, which optionally includes one or more of the first through sixth examples, the diagnosis determination logic comprises a supervised machine learning model. In an eighth example, which optionally includes one or more of the first through seventh examples, wherein the supervised machine learning model comprises a random forest, support vector machine, Bayesian Decision List, linear regression, logistic regression, naïve Bayes, linear discriminant analysis, decision tree, k-nearest neighbor, or neural network. In a ninth example, which optionally includes one or more of the first through eighth examples, each of the at least three types of sensors, each comprise a sensor that detects different types of data from a user. In a tenth example, which optionally includes one or more of the first through ninth examples, the at least three types of sensors comprises at least 4, 5, 6, 7, 8, 9, 10, 11, 12, 13, 14, 15, 16, 17, 18, 19, or 20 types of sensors. In an eleventh example, which optionally includes one or more of the first through tenth examples, the diagnosis determination logic is pre-trained using data output from the at least three types of sensors on patients with and without mental health conditions.

### Experimental Data

The following set of experimental data is provided to better illustrate the claimed invention and is not intended to be interpreted as limiting the scope.

Example mental health evaluation using a multimodal product fusion model, such as the product fusion models described herein, is described below to identify symptoms of mood disorders using audio, video and text collected using a smartphone app. The mood disorders include depression, anxiety, and anhedonia, which are predicted using the multimodal product fusion model. Unimodal encoders were used to learn unimodal embeddings for each modality and then an outer product of audio, video, and text embeddings was generated to capture individual features as well as higher order interactions. These methods were applied to a dataset collected by a smartphone application on 3002 participants across up to three recording sessions. The product fusion method demonstrated better mental health classification performance compared to existing methods that employed unimodal classification.

### Dataset

The data used in this experiment was collected remotely through an interactive smartphone application that was available to the U.S. general population through Google Play and Apple App Store under IRB approval. (1) Demographic Variables and Health History (2) Self-reported clinical scales including Patient Health Questionnaire-9 (PHQ-9), Generalized Anxiety Disorder-7 (GAD-7) and Snaith Hamilton Pleasure Scale (SHAPS) and (3) Video recorded vocal expression activities (where participants were asked to record videos of their faces while responding verbally to prompts) were collected on each of 3002 unique participants. The entire set of video tasks took less than five minutes, and participants could provide data up to three times (across 4 weeks), for a total of 3 sessions (not all participants completed 3 sessions).

### Feature Extraction and Quality Control

Audio, video and text features were extracted to perform model building. However, since this data was collected without human supervision, a rigorous quality control procedure was performed to reduce noise.

### Feature Extraction

Audio: These represent the acoustic information in the response. Each audio file was denoised, and unvoiced segments were removed. A total of 123 audio features (including prosodic, glottal and spectral) were extracted at a resolution of 0.1 seconds. In particular, for each audio file, 123 audio features were extracted from the voiced segments at a resolution of 0.1 seconds, including prosodic (Pause rate, speaking rate etc.), glottal (Normalised Amplitude Quotient, Quasi-Open-Quotient etc.), spectral (Mel-frequency cepstral coefficients, Spectral Centroid, Spectral flux, Mel-frequency cepstral coefficient spectrograms etc.) and chroma (Chroma Spectogram) features.

Video: These represent the facial expression information in the response. For each video, 3D facial landmarks were computed at a resolution of 0.1 seconds. From these, 22 Facial Action Units were computed for modeling. In particular, for each video file, 22 Facial Action Unit features were extracted. These were derived from 3D facial landmarks which were computed at a resolution of 0.1 seconds. This was in contrast to prior approaches where 2D facial landmarks have been primarily used. Through these experiments, the inventors identified that 3D facial landmarks were much more robust to noise than 2D facial landmarks, thus making these more effective for remote data collection and analysis.

Text: These represent the linguistic information in the response. Each audio file was transcribed using Google Speech-to-Text and 52 text features were computed including affective features, word polarity and word embeddings. In particular, for each file, 52 text features were extracted including affect based features viz. arousal, valence and dominance rating for each word using Warriner Affective Ratings, polarity for each word using TextBlob, contextual features such as word embeddings using doc2vec, etc.

### Quality Control

In contrast to prior approaches, where the data was collected under clinical supervision (e.g. the DAIC-WOZ dataset), the data used herein was collected remotely on consumer smartphones. Consequently, this data could have more noise that needed to be addressed before modeling. There were two broad sources of noise: (1) Noisy medium (e.g. background audio noise, video failures and illegible speech) and (2) Insincere participants (e.g. participant answering “blah” to all prompts). Using the metadata, scales and extracted features, quality control flags were implemented to screen participants. These included flags on (1) Video frame capture failures (poor lighting conditions) (2) Missing transcriptions (excessive background noise or multiple persons speaking) (3) Illegible speech and (4) Inconsistent responses between similar questions of clinical scales, among other flags. Out of 6020 collected sessions, 1999 passed this stage. The developed flags can be pre-built into the app for data collection. A multimodal machine learning approach was implemented to classify symptoms of mood disorders. Specifically, the audio, video and textual modalities for the 1999 sessions were used as input, and performed three classification problems to predict binary outcome labels related to the presence of symptoms of (1) depression (total PHQ-9 score >9), (2) anxiety (total GAD-7 score >9), and (3) anhedonia (total SHAPS score >25)). In this dataset, 71.4% of participants had symptoms of depression, 57.8% of participants had symptoms of anxiety and 67.3% of participants had symptoms of anhedonia. The dataset described above is much larger than the DAIC-WOZ dataset in AVEC 2019 (N=275) and also contained a higher percentage of individuals with depression symptoms (our dataset=71.4%, AVEC=25%).

### Experiments and Results

The product fusion multimodal method outperformed state of the art work employing unimodal embeddings: BiLSTM-Static Attention (Ray et al. Multi-level attention network using text, audio, and video for depression prediction. In Proceedings of the 9th International on Audio/Visual Emotion Challenge and Workshop, AVEC '19, pp. 81-88, New York, NY, USA, 2019) in multimodal classification of symptoms across at least two domains tested: depression (PHQ-9) and anxiety (using GAD-7). Two different aspects of performance were compared: First the overall classification performance across the two scales (using median test F1 score as the metric) was compared and the results are shown in Table 1. The product fusion method (indicated as LSTM+Tensor Fusion in the tables below) performed better compared to the other method across PHQ-9 and GAD-7 scales. Next, models with each of the modalities were built and the performance of the multimodal model vs the best unimodal model (using the percentage difference in median test F1 score between multimodal and best unimodal) was compared for the different approaches and across the two scales (Table 2).

TABLE 1
Multimodal classification of mood disorder
symptoms: Median Test F1 Score

| Method (Feature Encoding + Fusion) | PHQ-9 | GAD-7 |
|---|---|---|
| BiLSTM + Static Attention | 0.625 | 0.5716 |
| LSTM + Tensor Fusion | 0.632 | 0.601 |

TABLE 2
Percentage Difference in Median Test F1 Score
between trimodal and best unimodal model

| Scale | Method (Feature Encoding + Fusion) | Percentage Difference |
|---|---|---|
| PHQ-9 | BiLSTM + Static Attention | 0 |
| | LSTM + Tensor Fusion | 0.16 |
| GAD-7 | BiLSTM + Static Attention | −0.84 |
| | LSTM + Tensor Fusion | 0.16 |

As evidenced above, the multimodal product fusion method showed a notable increase in performance in the multimodal case whereas the other approach showed no increase (or sometimes decrease). This demonstrates that the multimodal product fusion method is able to efficiently capture the interaction information across different modalities.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### Selected Embodiments

Although the above description and the attached claims disclose a number of embodiments of the present invention, other alternative aspects of the invention are disclosed in the following further embodiments.

Embodiment 1: A device, comprising: a first modality processing logic to process a first data modality from a first type of sensor to output a first data representation comprising a first set of features; a second modality processing logic to process a second data modality from a second type of sensor to output a second data representation comprising a second set of features; modality combination logic to process the first and second data representations to output a combined data representation comprising products of the first and second set of features; relevance determination logic to identify the relevance of each of the products of the first and second features to a mental health diagnosis; and diagnosis determination logic to determine a mental health diagnosis based on the relevance of the products of the first and second set of features to the mental health diagnosis.
Embodiment 2: The device of embodiment 1, wherein the first and second sensor type each comprise one of: a camera, a microphone, a MRI scanner, a user interface, a keyboard, an EEG detector, or a plate reader.
Embodiment 3: The device of embodiment 1, wherein the first and second modality processing logic each further comprise a first and second modality preprocessing logic.
Embodiment 4: The device of embodiment 3, wherein the first and second modality preprocessing logic comprises a feature dimensionality reduction model.
Embodiment 5: The device of embodiment 1, wherein the first and second modality processing logic comprises at least one of: a feed-forward neural network, a convolutional neural network, a long short-term memory network (LSTM), or a transformer.
Embodiment 6: The device of embodiment 1, wherein the modality combination logic comprises a tensor fusion model, the tensor fusion model configured to generate the combined data representation based on an outer product of all of the first set of features and all of the second set of features.
Embodiment 7: The device of embodiment 1, wherein the relevance determination logic comprises at least one of a feed-forward neural network, or an attention model.
Embodiment 8: The device of embodiment 1, wherein the diagnosis determination logic comprises a supervised machine learning model.
Embodiment 9: The device of embodiment 8, wherein the supervised machine learning model comprises a random forest, support vector machine, Bayesian Decision List, linear regression, logistic regression, naïve Bayes, linear discriminant analysis, decision tree, k-nearest neighbor, or neural network.
Embodiment 10: The device of embodiment 8, wherein the supervised machine learning model is trained using responses to clinical questionnaires as the outcome label.
Embodiment 11: The device of embodiment 1, wherein the first and second modality processing logic is trained separately from the relevance determination logic.
Embodiment 12: The device of embodiment 1, wherein the first and second modality processing logic is trained jointly with the relevance determination logic.
Embodiment 13: The device of embodiment 2, wherein the camera is a three dimensional camera.
Embodiment 14: The device of embodiment 1, wherein the mental health diagnosis comprises at least one of: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, or a dementia.
Embodiment 15: The device of embodiment 1, wherein the mental health diagnosis comprises a quantitative assessment of a severity of the mental health disorder.
Embodiment 16: A device comprising: a first modality processing logic to process data output from a first type of sensor to output a first set of features; a second modality processing logic to process data output from a second type of sensor to output a second set of features; a product determination logic to determine a product of the first and second set of features; a diagnostic relevance interaction logic to identify a relevance of each of the products of the first and second set of features to a mental health diagnosis; and a diagnosis determination logic to determine a mental health diagnosis based on the diagnostic relevance of each of the products of the first and second set of features.
Embodiment 17: The device of embodiment 16, further comprising a third modality processing logic to process data output from a third type of sensor to output a third set of features.
Embodiment 18: The device of embodiment 17, wherein the product of the first and second set of features comprises the product of the first, second, and third set of features.
Embodiment 19: The device of embodiment 18, wherein the relevance of the first and second set of features comprises the relevance of the first, second, and third set of features.
Embodiment 20: The device of embodiment 19, wherein the diagnostic relevance of each of the products of the first and second set of feature further comprises the diagnostic relevance of each of the products of the first, second, and third set of features.
Embodiment 21: The device of embodiment 17, wherein the first type of sensor comprises a camera, the second type of sensor comprises a microphone, and the third type of sensor comprises a user interface configured to receive textual user input.
Embodiment 22: The device of embodiment 21, wherein the first set of features comprises facial features, the second set of features comprises voice features, and the third set of features comprises textual features.
Embodiment 23: A computing device comprising: a memory containing machine readable medium comprising machine executable code having stored thereon instructions; and a control system coupled to the memory comprising one or more processors, the control system configured to execute the machine executable code to cause the control system to: receive a first set of data comprising a first data modality output from a first type of sensor; receive a second set of data comprising a second data modality from a second type of sensor; receive a third set of data comprising a third data modality output from a third type of sensor; process the first set of data with a first model to output a first data representation comprising a first feature set; process the second set of data with a second model to output a second data representation comprising a second feature set; process the third set of data with a third model to output a third data representation comprising a third feature set; and process the first, the second, and the third data representations with a product model to output a set of combination features, wherein each of the set of combination features comprising products of the first, second, and third feature set; and process the set of combination features using a fourth model to output a combined data representation.
Embodiment 24: The computing device of embodiment 23, wherein the first, second, and third type of sensor each comprise one of: a camera, a microphone, a MRI scanner, a user interface, a keyboard, an EEG detector, or a plate reader.
Embodiment 25: The computing device of embodiment 23, wherein the first data modality comprises image data, video data, three dimensional video data, audio data, MRI data, text strings, EEG data, gene expression data, ELISA data, or PCR data.
Embodiment 26: The computing device of claim 23, wherein the camera comprises a three dimensional camera.
Embodiment 27: The computing device of embodiment 23, wherein the product model is a tensor fusion model.
Embodiment 28: The computing device of embodiment 23, wherein the mental health classification comprises: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, or a dementia.
Embodiment 29: The computing device of embodiment 23, wherein process the set of combination features using a fourth model further comprises first processing the set of combination features using an attention model.
Embodiment 30: The computing device of embodiment 23, wherein the first, second, and third data representation comprise feature vectors.
Embodiment 31: The computing device of embodiment 23, wherein the first, second, and third data modality each comprise a unique data format.
Embodiment 32: The computing device of embodiment 23, wherein the first data representation comprises an output from a convolution neural network, long short-term memory network, transformer, or a feed forward neural network.
Embodiment 34: The computing device of embodiment 23, wherein the first model comprises a feed-forward neural network, a convolutional neural network, a long short-term memory network (LSTM), or a transformer.
Embodiment 35: The computing device of embodiment 23, wherein the fourth model comprises a feed-forward neural network.
Embodiment 36: The computing device of embodiment 23, wherein the control system is further configured to execute the machine executable code to cause the control system to process the combined data representation with a supervised machine learning model to output a mental health classification of a patient.
Embodiment 37: The computing device of embodiment 23, wherein the first, second and third models are trained separately from the fourth model.
Embodiment 38: The computing device of embodiment 23, wherein the first, second, third and fourth models are trained jointly.
Embodiment 39: The computing device of embodiment 36, wherein the supervised machine learning model comprises a random forest, support vector machine, Bayesian Decision List, linear regression, logistic regression, naïve Bayes, linear discriminant analysis, decision tree, k-nearest neighbor, or neural network.
Embodiment 40: A computing device comprising: a memory containing machine readable medium comprising machine executable code having stored thereon instructions; and a control system coupled to the memory comprising one or more processors, the control system configured to execute the machine executable code to cause the control system to: receive a first set of data comprising a first data modality output from a first type of sensor; receive a second set of data comprising a second data modality from a second type of sensor; receive a third set of data comprising a third data modality output from a third type of sensor; process all of the first set of data with a first model to output a first data representation comprising a first feature set; process all of the second set of data with a second model to output a second data representation comprising a second feature set; process all of the third set of data with a third model to output a third data representation comprising a third feature set; and process all of the first, all of the second, and all of the third data representations with a product model to output a set of combination features, wherein each of the set of combination features comprising products of the first, second, and third feature set; and process the set of combination features using a fourth model to output a combined data representation.
Embodiment 41: A device, comprising: a modality processing logic to process data output from at least three types of sensors to output a set of data representations for each of the at least three types of sensors, wherein each of the set of data representations comprises a vector comprising a set of features; modality combination logic to process the set of data representations to output a combined data representation comprising an outer product of the set of data representations; relevance determination logic to identify the relevance of each of the outer product to a mental health diagnosis; and diagnosis determination logic to determine a mental health diagnosis based on the relevance of the outer product to the mental health diagnosis.
Embodiment 42: The device of embodiment 41, wherein the at least three types of sensors each comprise at least one of: a camera, a microphone, a MRI scanner, a user interface, a keyboard, an EEG detector, or a plate reader.
Embodiment 43: The device of embodiment 41, wherein the modality processing logic further comprises a preprocessing logic.
Embodiment 44: The device of embodiment 43, wherein the preprocessing logic comprises a feature dimensionality reduction model.
Embodiment 45: The device of embodiment 41, wherein the modality processing logic comprises at least one of: a feed-forward neural network, a convolutional neural network, a long short-term memory network (LSTM), or a transformer.
Embodiment 46: The device of embodiment 41, wherein the modality combination logic comprises a tensor fusion model.
Embodiment 47: The device of embodiment 41, wherein the relevance determination logic comprises at least one of a feed-forward neural network, or an attention model.
Embodiment 48: The device of embodiment 41, wherein the diagnosis determination logic comprises a supervised machine learning model.
Embodiment 49: The device of embodiment 48, wherein the supervised machine learning model comprises a random forest, support vector machine, Bayesian Decision List, linear regression, logistic regression, naïve Bayes, linear discriminant analysis, decision tree, k-nearest neighbor, or neural network.
Embodiment 50: The device of embodiment 41, wherein each of the at least three types of sensors, each comprise a sensor that detects different types of data from a user.
Embodiment 51: The device of embodiment 41, wherein the at least three types of sensors comprises at least 4, 5, 6, 7, 8, 9, 10, 11, 12, 13, 14, 15, 16, 17, 18, 19, or 20 types of sensors.
Embodiment 52: The device of embodiment 41, wherein the diagnosis determination logic is pre-trained using data output from the at least three types of sensors on patients with and without mental health conditions.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A device, comprising:
a first modality processing logic configured to process all of a first data modality received from a first type of sensor to output a first data representation comprising a first set of features;
a second modality processing logic configured to process all of a second data modality received from a second type of sensor to output a second data representation comprising a second set of features;
modality combination logic configured to process the first and second data representations to output a combined data representation comprising products of the first and second sets of features;
relevance determination logic configured to identify the relevance of each of the products of the first and second features to a mental health diagnosis; and
diagnosis determination logic configured to determine a mental health diagnosis based on the relevance of the products of the first and second sets of features to the mental health diagnosis.

2. The device of claim 1, wherein the first and second sensor type each comprise one of: a camera, a microphone, a MRI scanner, a user interface, a keyboard, an EEG detector, or a plate reader.

3. The device of claim 1, wherein the first and second modality processing logic each further comprise a first and second modality preprocessing logic.

4. The device of claim 1, wherein the relevance determination logic is further configured to:
generate a low dimensional representation of the combined data representation by simplifying the products of the first and second sets of features; and
identify the relevance of each of the simplified products in the low dimensional representation of the combined data representation to a mental health diagnosis.

5. The device of claim 4, wherein the low dimensional representation of the combined data representation is generated using at least one of: a feed-forward neural network, a convolutional neural network, a long short-term memory network (LSTM), and a transformer.

6. The device of claim 1, wherein the modality combination logic comprises a tensor fusion model, the tensor fusion model configured to generate the combined data representation based on an outer product of all of the first set of features and all of the second set of features.

7. The device of claim 1, wherein the relevance determination logic comprises at least one of a feed-forward neural network, or an attention model.

8. The device of claim 1, wherein the diagnosis determination logic comprises a supervised machine learning model.

9. The device of claim 8, wherein the supervised machine learning model comprises a random forest, support vector machine, Bayesian Decision List, linear regression, logistic regression, naïve Bayes, linear discriminant analysis, decision tree, k-nearest neighbor, or neural network.

10. The device of claim 8, wherein the supervised machine learning model is trained using responses to clinical questionnaires as the outcome label.

11. The device of claim 1, wherein the first and second modality processing logic is trained separately from the relevance determination logic.

12. The device of claim 1, wherein the first and second modality processing logic is trained jointly with the relevance determination logic.

13. The device of claim 2, wherein the camera is a three dimensional camera.

14. The device of claim 1, wherein the mental health diagnosis comprises at least one of: a psychiatric disorder, a depression, a schizophrenia, an anxiety, a panic disorder, a borderline personality disorder, an obsessive compulsive disorder, a post-traumatic stress disorder, an autism spectrum disorder, a mood disorder in epilepsy, a personality disorder, a cognitive change associated with chemotherapy, an attention deficient hyperactivity disorder (ADHD), a neurodevelopmental disorder, a neurodegenerative disorder, an Alzheimer's disease, or a dementia.

15. The device of claim 1, wherein the mental health diagnosis comprises a quantitative assessment of a severity of the mental health disorder.

16. A device comprising:
a first modality processing logic configured to process data received from a first type of sensor to output a first set of features;
a second modality processing logic configured to process data received from a second type of sensor to output a second set of features;
a product determination logic configured to determine products of the first and second sets of features;
a diagnostic relevance interaction logic configured to:
generate low dimensional representations of the products of the first and second sets of features; and
identify a relevance of each of the low dimensional representations to a mental health diagnosis; and
a diagnosis determination logic configured to determine a mental health diagnosis based on the diagnostic relevance of each of the low dimensional representations.

17. The device of claim 16, further comprising a third modality processing logic to process data received from a third type of sensor to output a third set of features, wherein the product determination logic is further configured to determine products of the first, second, and third sets of features, wherein the low dimensional representations correspond to the products of the first, second, and third sets of features.

18. The device of claim 17, wherein the product of the first and second sets of features comprises the product of the first, second, and third set of features.

19. The device of claim 18, wherein the relevance of the first and second sets of features comprises the relevance of the first, second, and third set of features.

20. The device of claim 19, wherein the diagnostic relevance interaction logic is further configured to:
determine whether a total number of modality types included in the products of the first and second sets of features is greater than a predetermined number; and
in response to determining that the total number of modality types included in the products of the first and second sets of features is greater than a predetermined number, generate the low dimensional representations.

21. The device of claim 17, wherein the first type of sensor comprises a camera, the second type of sensor comprises a microphone, and the third type of sensor comprises a user interface configured to receive textual user input.

22. The device of claim 21, wherein the first set of features comprises facial features, the second set of features comprises voice features, and the third set of features comprises textual features.
